(19) United States
(12) Patent Application Publication
(10) Pub. No.: US 2011/0152957 A1
(43) Pub. Date: Jun. 23, 2011

(54) CHAOS-BASED DETECTION OF ATRIAL FIBRILLATION USING AN IMPLANTABLE MEDICAL DEVICE

(76) Inventor: Cem Shaquer, Los Gatos, CA (US)

(21) Appl. No.: 12/643,867

(22) Filed: Dec. 21, 2009

(51) Int. Cl. A61N 1/365; A61B 5/046; A61N 1/39

(52) U.S. Cl. 607/5; 600/516; 607/14

(57) ABSTRACT

Techniques are provided for detecting atrial fibrillation (AF) based on variations in ventricular intervals detected by a pacemaker, implantable cardioverter-defibrillator (ICD) or implantable cardiac monitor (ICM). In one example, ventricular beats are detected and intervals between the ventricular beats are measured, such as RR intervals. Irregular ventricular beats are identified, including ectopic beats, bigeminal beats, and the like. The degree of variability within the ventricular intervals is then determined while excluding any intervals associated with irregular beats. AF is then detected based on the degree of variability. That is, AF is detected based on variability occurring within ventricular intervals after ectopic beats and other irregular beats have been eliminated, thus mitigating detection problems that might arise if the variability were instead calculated based on all ventricular beat intervals. Techniques are also described herein for distinguishing AF from sinus tachycardia, which can also cause a high degree of variability in RR intervals.

### FIELD OF THE INVENTION

The invention generally relates to implantable medical devices such as pacemakers, implantable cardioverter-defibrillators (ICDs) and implantable cardiac monitors (ICMs) and, in particular, to techniques for detecting and distinguishing atrial fibrillation (AF) and other supraventricular arrhythmias using such devices.

### BACKGROUND OF THE INVENTION

A pacemaker is an implantable medical device that recognizes various arrhythmias, including supraventricular arrhythmias such as atrial tachycardia (AT), atrial fibrillation (AF), and sinus tachycardia (ST) and ventricular arrhythmias such as ventricular tachycardia (VT) and ventricular fibrillation (VF). The pacemaker delivers electrical pacing pulses to the heart in an effort to prevent, suppress or remedy such arrhythmias. An ICD is an implantable device that is additionally capable of delivering high voltage electrical shocks to terminate AF or VF. An ICM is a diagnostic tool implanted beneath patient skin, which monitors the patients electrocardiogram (ECG) and rhythm continuously and records ECGs for episodes of arrhythmias, either triggered via external activation by the patient during symptoms or by automatic detection of cardiac arrhythmias, such as asystole, bradycardia, tachycardia, atrial fibrillation, atrial tachycardia, atrial flutter, etc.

During AF, the atria of the heart beat chaotically. Patients often feel heart palpitations, fainting, dizziness, weakness, shortness of breath and angina pectoris (chest pain caused by a reduced blood supply to the heart muscle). Although not life threatening immediately, AF increases the mortality risk in elder patients and increases the risk of a debilitating stroke about three times in the patients >75 yrs of age. The prevalence of AF is about 2.2 million in the US. Moreover, the irregular beating of the atria during AF interferes with the proper hemodynamic function of the heart by preventing the ventricles from filling properly. As a result, optimal ventricular pressure is not achieved during each heartbeat and overall cardiac performance is degraded, i.e. the ventricles do not efficiently pump blood into the circulatory system. The ventricular rate may become somewhat erratic as well, due to conduction from the atria to the ventricles, possibly triggering a ventricular tachyarrhythmia. Furthermore, during AF, blood tends to pool in the atrial chambers, increasing the risk of a blood clot forming. Once formed, a blood clot can travel from the heart into the bloodstream and through the body, potentially becoming lodged in an artery, possibly causing a pulmonary embolism or stroke. Hence, steps are preferably taken to prevent the occurrence AF and, should an episode of AF nevertheless occur, it is deemed advisable, at least conventionally, to terminate the episode as soon as possible via one or more cardioversion shocks.

In dual-chamber and tri-chamber pacemakers and ICDs (i.e. devices that have at least one atrial lead), atrial activity is monitored by the device using the atrial lead. The atrial information is used by the device for a range of functions including discrimination of supraventricular arrhythmias from ventricular arrhythmias, detection of AT/AF (which are specific types of supraventricular arrhythmias), as well as generation of AT/AF burden diagnostics. In devices without an atrial lead, however, the lack of direct atrial information requires the device to detect AT/AF and/or discriminate supraventricular arrhythmias from ventricular arrhythmias based only on data sensed by ventricular leads. Depending upon the sensitivity of the ventricular channel signals, at least some atrial depolarization events (i.e. P-waves) may appear within the ventricular signal. However, such events are typically of too low a magnitude to allow for reliable detection of AF or other supraventricular arrhythmias.

Hence, techniques have been developed for detecting AF based on an analysis of the much higher magnitude ventricular depolarization events (i.e. R-waves or QRS-complexes) appearing within the ventricular channel intracardiac electrogram (IEGM). In particular, variations in the intervals between R-waves (i.e. RR variability) has been exploited to detect AF and to discriminate among different types of supraventricular arrhythmias (e.g. AF, AT, atrial flutter, etc.). See, for example, Lorentz scatter plot-based techniques set forth in U.S. Pat. No. 7,031,765 to Ritscher et al. U.S. Pat. No. 7,412,282 of Houben also presents techniques for, inter alia, detecting AF based on ventricular signals.

Similar problems arise when detecting AF using an ICM. The ICM, which is implanted subcutaneously, continuously monitors electrical signals to detect cardiac arrhythmias such as AF and to record the cardiac signals during the arrhythmia, as well as information pertaining to other important events. (Note that the cardiac signal data recorded by a pacemaker or ICD differs somewhat from the data recorded by an ICM. The data recorded by a pacemaker/ICD using intracardiac electrodes is referred to as an intracardiac electrogram (IEGM). The cardiac signal data recorded by ICM using subcutaneous electrodes is referred to as a subcutaneous electrocardiogram (EGM).)

The EGM recording in an ICM occurs either by external triggering, which is typically is activated by the patient upon feeling of symptoms of an arrhythmia, or by automatic detection of an arrhythmia or other event by the implanted device. Although ICMs are not equipped to deliver stimulation therapy to the heart, such devices can nevertheless detect AF and other arrhythmias and present the physician with the subcutaneous EGM recorded during the episode for diagnosis of cardiac arrhythmias with unexplained symptoms as well as long term monitoring of AF patients post-ablation to detect recurrence of asymptomatic episodes. Furthermore, ICMs (or implantable loop recorders) can be a useful diagnostic tool to aid the physician in controlling warfarin therapy in post-ablation patients (where warfarin is an anticoagulant.) Frequent episodes of AF within a patient may warrant implant of an ICD so that cardioversion shocks can then be delivered automatically in response to further episodes of AF.

Due to use of subcutaneous electrodes positioned beneath the skin, the P-waves appearing in the ICM EGMs are typically an order of magnitude smaller than that of the R-waves. See, FIG. 1, which provides an exemplary subcutaneous EGM 2 including R-waves 4, T-waves 6 (i.e. ventricular repolarization complexes), as well as low-magnitude P-waves 8. As can be seen, the P-waves are quite small compared to the other features of the EGM and hence can be easily obscured by noise, including artifacts caused by patient motion, respiration, skeletal muscle movement, etc. As such, AF is preferably detected based on the R-waves of the EGM, and so the aforementioned RR variability-based techniques are potentially advantageous.

Although RR analysis techniques are promising for use in ICMs and in pacemaker/ICDs lacking atrial leads, problems remain. For example, the presence of ectopic beats, bigeminal beats, trigeminal beats, or other irregular ventricular beats can interfere with the reliable detection of AF. Also, it can be difficult to distinguish AF from sinus tachycardia, as both can cause similar changes to RR variability.

Accordingly, it would be desirable to provide improved RR analysis techniques for detecting AF or for discriminating among different types of supraventricular arrhythmias using ICMs or pacemaker/ICDs, and it is to that end that aspects of the invention are directed.

### SUMMARY OF THE INVENTION

In accordance with an exemplary embodiment of the invention, a method is provided for use with an implantable medical device such as a pacemaker, ICD or ICM for detecting AF or other supraventricular arrhythmias. Ventricular beats are detected and intervals between the ventricular beats are measured. Irregular ventricular beats are identified, such as ectopic beats, bigeminal beats, and the like. A degree of variability within the ventricular intervals is determined while excluding intervals associated with irregular beats. Then, supraventricular arrhythmia is detected based on the degree of variability. That is, supraventricular arrhythmias such as AF are detected based on the variability within ventricular intervals after ectopic beats and other irregular beats have been eliminated, thus mitigating detection problems that might arise if the variability were instead calculated based on all ventricular beat intervals.

In an illustrative embodiment, ventricular events are detected based on R-waves sensed within an IEGM or subcutaneous EGM, and RR intervals are measured. Ectopic beats, bigeminal beats and trigeminal beats are then identified based on the patterns these irregular beats cause within the RR or dRR intervals. The RR or dRR intervals associated with individual irregular ventricular beats are then eliminated. The degree of variability in the remaining RR intervals is evaluated using a chaos-based technique. In one particular example, dRR values are derived from the RR intervals after RR intervals associated with irregular beats (such as PACs, PVCs, bigeminy, trigeminy etc.) are eliminated. An average dRR value is calculated and any dRR values deviating significantly from the average are identified. A numerical value is then generated that is representative of the number of dRR values that deviate significantly from the dRR average. The numerical value is thereby representative of the degree of chaos within the intervals. If the numerical value exceeds a threshold indicative of a possible episode of AF, the implantable device further determines whether the episode was subject to sudden onset. If so, the episode is deemed to be AF. Otherwise, it is deemed to be normal sinus rhythm, ST or other non-AF supraventricular arrhythmias. Conceptually, the variability evaluation procedure is equivalent to generating a Poincare plot based on dRR intervals, then identifying points within the Poincare plot outside of a central portion. The region outside the central portion corresponds to AF/ST.

The degree of variability within the ventricular intervals may also be used to distinguish among a variety of supraventricular arrhythmias. In particular, atrial flutter and junctional rhythms are identified based on tightly varying RR intervals. Premature atrial contractions (PACs) and premature ventricular contractions (PVCs) are identified based on sudden changes in the RR intervals, without substantially variability. Bigeminal and trigeminal rhythms are identified based on substantially cyclic variations in RR intervals. Sinus tachycardia is identified, as noted, based on a high degree RR variability that does not arise suddenly. In contrast, AF is identified based on a high degree RR variability that arises comparatively suddenly. Hence, AF and ST are distinguished from one another despite both providing a high degree of RR variability.

Thus, various improved techniques are provided for detecting AF and for discriminating among different types of supraventricular arrhythmias using ICMs or pacemaker/ICDs.

### BRIEF DESCRIPTION OF THE DRAWINGS

Further features and advantages of the invention may be more readily understood by reference to the following description taken in conjunction with the accompanying drawings, in which:

FIG. 1 is a graph illustrating an exemplary EGM recorded by an ICM, and particularly illustrating the relatively low magnitude P-waves appearing therein;

FIG. 2 provides an overview of components of an implantable medical system having a pacemaker or ICD equipped to detect AF based on RR variability subject to pre-elimination of ectopic beats and other irregular beats;

FIG. 3 provides an overview of components of an ICM also equipped to detect AF based on RR variability, also subject to pre-elimination of ectopic beats and other irregular beats;

FIG. 4 is a flow diagram providing an overview of a general method for detecting supraventricular arrhythmias, which may be performed by the systems of FIG. 2 or 3;

FIG. 5 is a block diagram summarizing pertinent hardware/firmware components for use in the systems of FIG. 2 or 3;

FIG. 6 is a graph illustrating exemplary EGM data processed by components such as those of FIG. 3;

FIG. 7 is a graph illustrating exemplary RR and dRR tachograms derived from the EGM of FIG. 6, which include artifacts due to PACs, bigeminy and AF;

FIG. 8 is a graph illustrating a portion of an EGM from which the tachograms of FIG. 7 were derived, and particularly illustrating a PAC;

FIG. 9 is a graph illustrating another portion of the EGM from which the tachograms of FIG. 7 were derived, and particularly illustrating various bigeminal events;

FIG. 10 is a graph illustrating another portion of the EGM from which the tachograms of FIG. 7 were derived, and particularly illustrating an episode of AF;

FIG. 11 includes a series of graphs illustrating Poincare plots generated based on the dRR tachogram of FIG. 7, and particularly illustrating an increasing amount of variability due to the onset of the episode of AF;

FIG. 12 includes a single graph illustrating one Poincare plot from the set of plots of FIG. 11, which particularly illustrates a +/−100 msec central portion for use in AF detection;

FIG. 13 is a table illustrating percentage data derived from the Poincare plot of FIG. 12, which particularly illustrates percentage increases occurring during the onset of the episode of AF;

FIG. 14 is a graph illustrating a portion of the tachogram of the dRR tachogram of FIG. 7, and particularly illustrating ectopic beats;

FIG. 15 is a graph illustrating a portion of the dRR tachogram of FIG. 7, and particularly illustrating bigeminal beats;

FIG. 16 is a graph illustrating a portion of the dRR tachogram of FIG. 7, and particularly illustrating an episode of AF;

FIG. 17 is a flow chart illustrating an exemplary method for filtering irregular beats from tachograms and for detecting AF, which may be performed by the devices of FIGS. 2 and 3;

FIG. 18 is a flow chart illustrating an exemplary method for calculating an interval average for use with technique of FIG. 17;

FIG. 19 is a flow chart illustrating an exemplary method for eliminating ectopic beats for use with technique of FIG. 17;

FIG. 20 is a flow chart illustrating an exemplary method for eliminating bigeminal beats for use with technique of FIG. 17;

FIG. 21 is a flow chart illustrating an exemplary method for searching for an indication of AF for use with technique of FIG. 17;

FIG. 22 is a graph illustrating just the dRR tachogram of FIG. 7, contrasting pre- and post-elimination versions of the tachogram;

FIG. 23 is a simplified, partly cutaway view, illustrating the pacer/ICD of FIG. 2;

FIG. 24 is a functional block diagram of the pacer/ICD of FIG. 23, illustrating basic circuit elements that provide cardioversion, defibrillation and/or pacing stimulation in the heart and particularly illustrating components for detecting AF;

FIG. 25 is a functional block diagram of the ICM of FIG. 3, illustrating components for detecting AF and recording subcutaneous EGM data.

### DESCRIPTION OF THE PREFERRED EMBODIMENTS

The following description includes the best mode presently contemplated for practicing the invention. The description is not to be taken in a limiting sense but is made merely for the purpose of describing the general principles of the invention. The scope of the invention should be ascertained with reference to the issued claims. In the description of the invention that follows, like numerals or reference designators will be used to refer to like parts or elements throughout. Both pacer/ICD and ICM implementations will be described.

### Overview of Implantable Pacer/ICD System

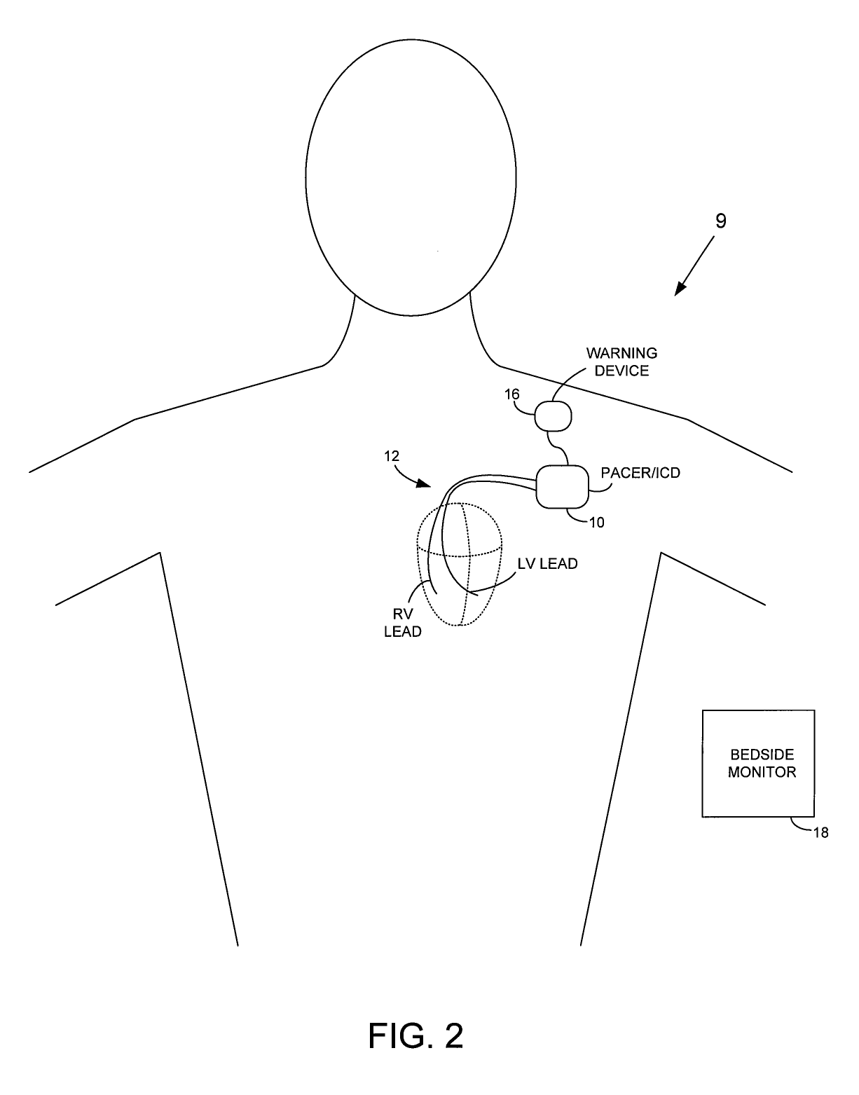

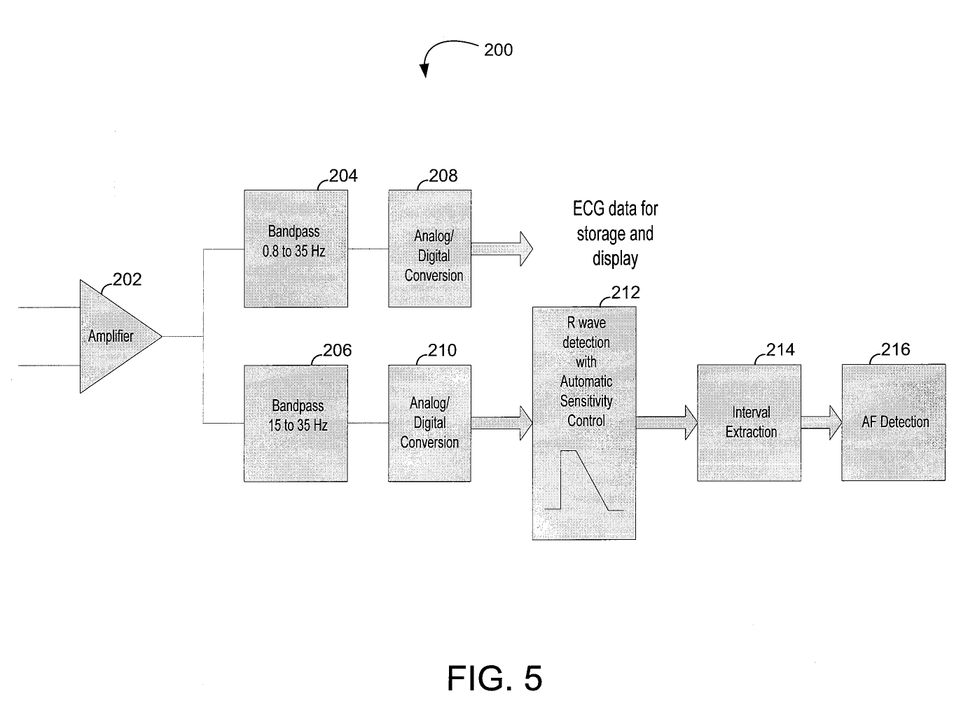

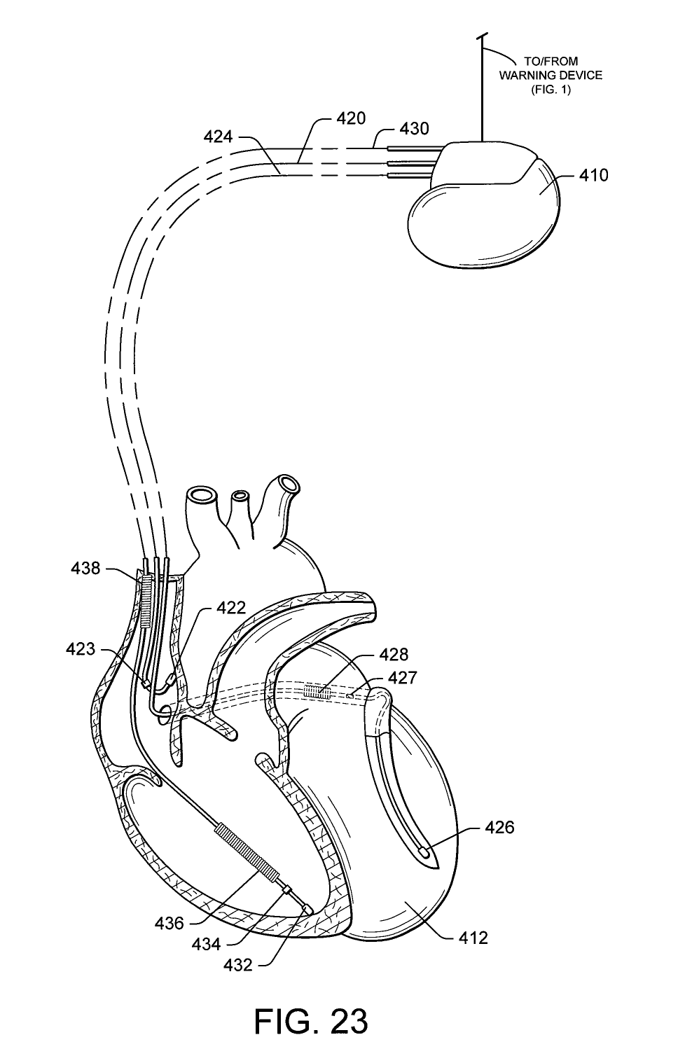

FIG. 2 provides a stylized representation of an implantable medical system 9 capable of various arrhythmias and delivering appropriate warnings or therapy. System 9 includes a pacer/ICD 10 or other cardiac stimulation device that incorporates internal components (shown individually in FIG. 5) for detecting arrhythmias, including AF and other supraventricular arrhythmias, based on IEGM signals sensed via a set of leads 12. In FIG. 2, stylized representations of right ventricular (RV) and left ventricular (LV) leads are shown. A more precise illustration of the location of the leads and their respective electrodes is provided in FIG. 23. Note that some pacer/ICDs also include an RA lead. When an RA lead is present, supraventricular arrhythmias are preferably detected based on atrial signals sensed by the RA lead, such that the RR interval-based techniques of the invention are typically not required. Nevertheless, the techniques of the invention can also be exploited within systems equipped one or more atrial leads to, e.g., confirm detection of supraventricular arrhythmias made using atrial leads before cardioversion shocks are delivered or to provide for a backup detection technique should the atrial lead fail.

Briefly, to detect supraventricular arrhythmias, the pacer/ICD processes ventricular signals received from the RV and LV leads to identify and eliminate irregular ventricular beats (e.g. ectopic beats and bigeminal beats), then analyzes RR intervals to detect AF and other supraventricular arrhythmias using a chaos-based technique, the details of which are presented below. If AF or other supraventricular arrhythmias are detected, appropriate therapy is delivered, such as delivery of cardioversion shocks via the leads to terminate the AF.

Techniques of delivering cardioversion shock therapy are set forth in U.S. Pat. No. 6,445,949 to Kroll. Preferably, procedures are implemented to reduce the pain associated with any cardioversion shocks. Techniques for pain reduction are set forth in the following patents and patent applications: U.S. Pat. Nos. 7,155,286, 7,231,255, and 7,480,531, each entitled “System and Method for Reducing Pain Associated with Cardioversion Shocks Generated by Implantable Cardiac Stimulation Devices”, to Kroll et al.; U.S. Pat. No. 5,830,236, to Mouchawar et al., entitled “System For Delivering Low Pain Therapeutic Electrical Waveforms To The Heart”; U.S. Pat. No. 5,906,633, also to Mouchawar et al., entitled “System for Delivering Rounded Low Pain Therapeutic Electrical Waveforms to the Heart”; U.S. Pat. No. 6,091,989 to Swerdlow et al., entitled “Method and Apparatus for Reduction of Pain from Electric Shock Therapies”; and U.S. Pat. No. 7,158,826 to Kroll et al., entitled “System and Method for Generating Pain Inhibition Pulses Using an Implantable Cardiac Stimulation Device.”

In patients where AF is common, it may be appropriate to deliver atrial overdrive pacing in an attempt to prevent AF. A particularly effective atrial overdrive pacing technique, referred to herein as dynamic atrial overdrive (DAO) pacing, is described in U.S. Pat. No. 6,519,493 to Florio et al., entitled “Methods and Apparatus for Overdrive Pacing Heart Tissue Using an Implantable Cardiac Stimulation Device”. With DAO, the overdrive pacing rate is controlled to remain generally uniform and, in the absence of a tachycardia, is adjusted upwardly or downwardly only occasionally in response to breakthrough sinus beats. The aggressiveness of overdrive pacing may be modulated by adjusting the overdrive pacing rate and related control parameters. See: U.S. Pat. Nos. 6,968,232 and 7,006,868, both of Florio et al., entitled “Method and Apparatus for using a Rest Mode Indicator to Automatically Adjust Control Parameters of an Implantable Cardiac Stimulation Device”; U.S. patent application Ser. No. 10/043,781, also of Florio et al., entitled “Method And Apparatus For Dynamically Adjusting A Non-Linear Overdrive Pacing Response Function”, filed Jan. 9, 2002; and U.S. Pat. No. 6,904,317, of Florio et al., entitled “Method And Apparatus For Dynamically Adjusting Overdrive Pacing Parameters”. Additionally, or in the alternative, the implantable system may be equipped with a drug pump (not shown) capable of the delivering any suitable drug therapy in an attempt to prevent or terminate AF.

Also, upon detection of AF or other supraventricular arrhythmias, warning signals may be delivered to warn the patient, using either an implantable warning device 16 or an external bedside monitor 18. Implantable warning device 16 may be a vibrating device or a “tickle” voltage device that, in either case, provides perceptible stimulation to the patient to alert the patient so that the patient may consult a physician. In some implementations, the implantable warning device is part of the pacer/ICD, rather than a separately implanted component. The bedside monitor may provide audible or visual alarm signals to alert the patient as well as textual or graphic displays. In addition, diagnostic information pertaining to AF and other arrhythmias may be transferred to the bedside monitor or stored within the pacer/ICD for subsequent transmission to an external programmer for review by a physician or other medical professional. The physician may then prescribe any other appropriate therapies to address the arrhythmias. The physician may also adjust the operation of the pacer/ICD to activate, deactivate or otherwise control any therapies that are automatically applied. The bedside monitor may be directly networked with a centralized computing system for immediately notifying the physician of any arrhythmias.

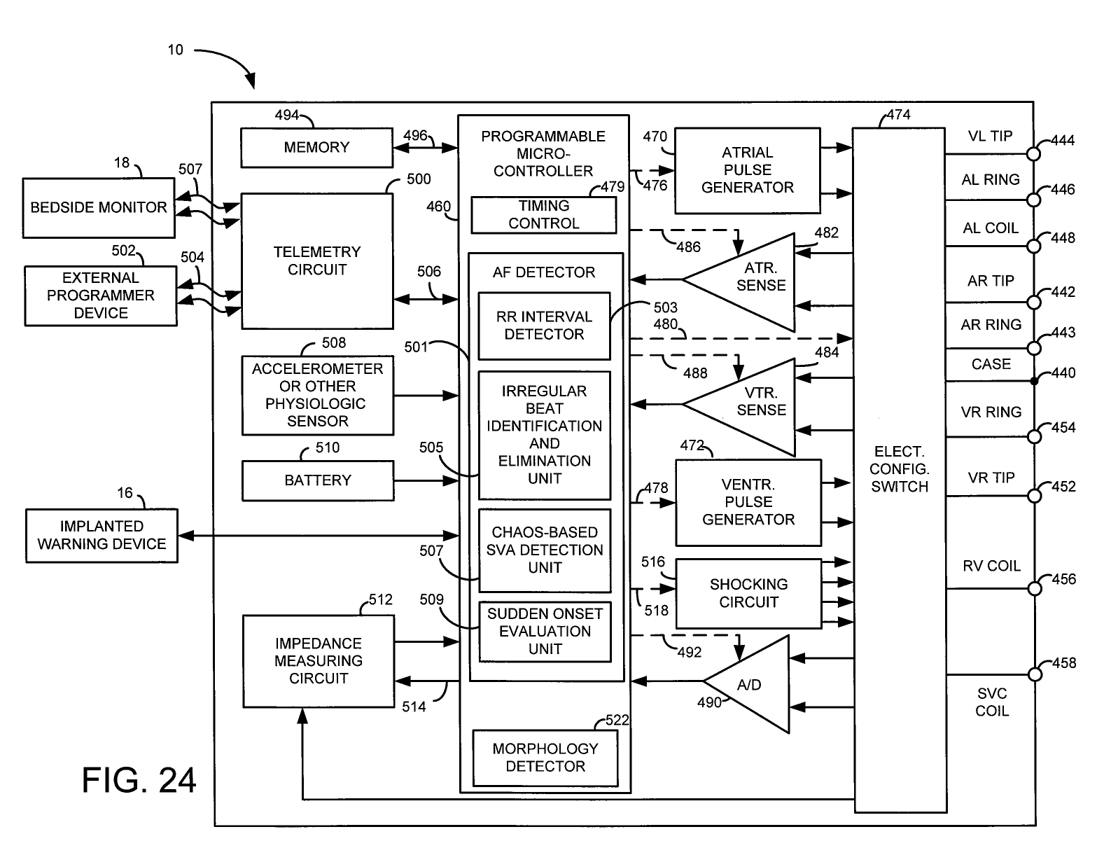

Preferably, in addition to the aforementioned functions, the device is capable of performing a wide range of other cardiac rhythm management functions (such detecting ventricular arrhythmias or delivering anti-tachycardia pacing (ATP) to prevent ventricular arrhythmias) and for delivering a wide range of other forms of electrical cardiac therapy (such as delivering defibrillation shocks in response to VF). This is discussed below with reference to FIG. 24.

Hence, FIG. 2 provides an overview of an implantable pacer/ICD system capable detecting AF and other supraventricular arrhythmias, controlling delivery of therapy in response thereto, generating warning signals and recording suitable diagnostic information. Embodiments may be implemented that do not necessarily perform all of these functions. For example, embodiments may be implemented that provide only detecting AF but not for delivering cardioversion therapy. Also, systems provided in accordance with the invention need not include all of the components shown in FIG. 2. In many cases, for example, the system will include only the pacer/ICD and its leads, with no implantable or external warning devices. Some implementations may employ an external monitor for generating warning signals but no implantable warning device, or vice versa. These are just a few exemplary embodiments. No attempt is made herein to describe all possible combinations of components that may be provided in accordance with the general principles of the invention. Also, note that internal signal transmission lines for interconnecting the various implanted components are shown in FIG. 2. Wireless signal transmission may alternatively be employed. Note also that the particular locations of the implanted components shown in FIG. 2 are merely illustrative and may not necessarily correspond to actual implant locations.

### Overview of ICM System

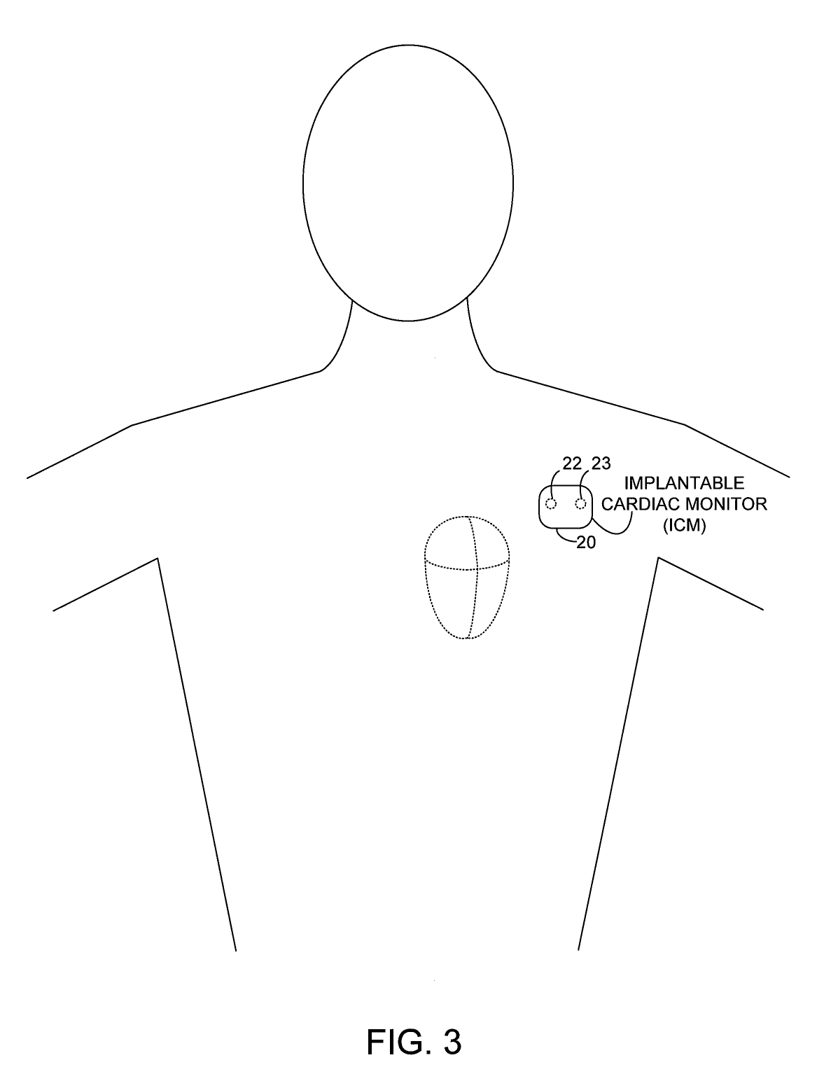

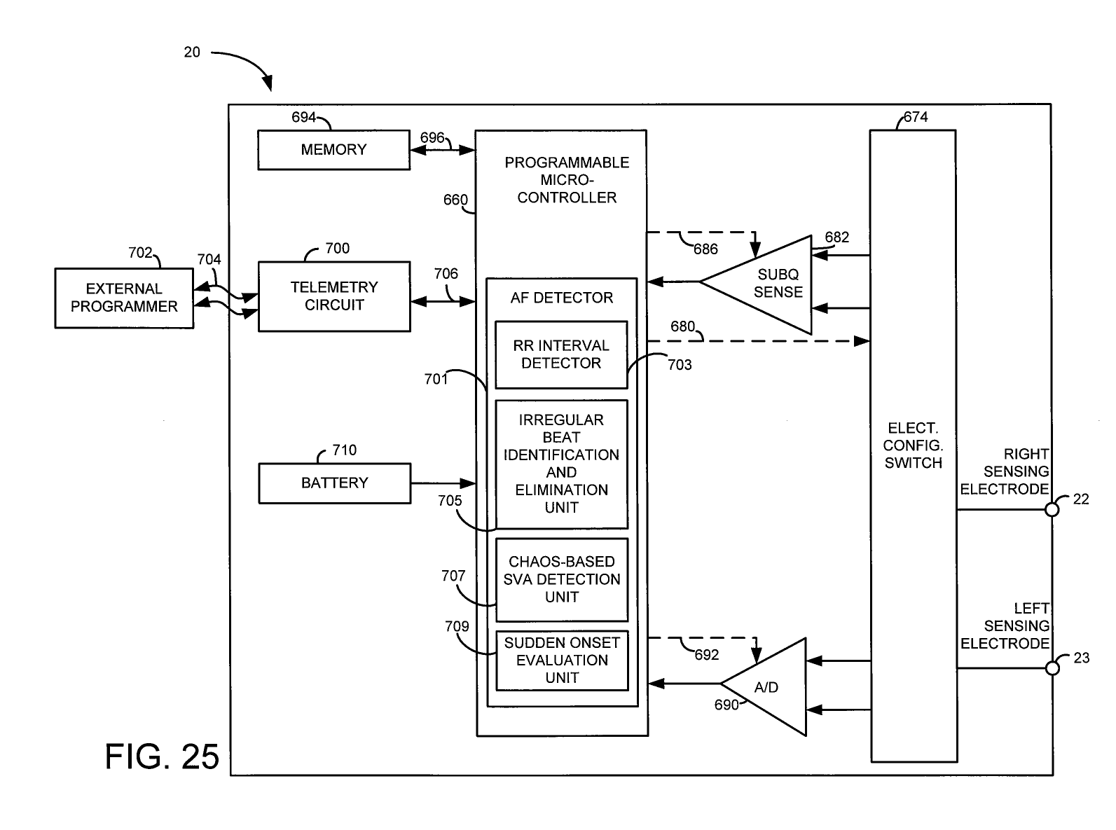

Referring next to FIG. 3, a broad overview of an ICM implementation is set forth provided. Briefly, FIG. 3 illustrates a subcutaneous ICM 20 having a pair of electrodes 22, 23 (shown in phantom lines) mounted to its housing for sensing subcutaneous (subQ) EGM signals within the chest of the patient. The ICM incorporates internal components (shown individually in FIG. 25) for detecting arrhythmias based on the subcutaneous EGM signals sensed via leads 22 and for recording suitable diagnostics information, such as the EGMs. Since the device is implanted subcutaneously, the subcutaneous EGM signals primarily include ventricular electrical signals.

If arrhythmias or other events of interest (such as episodes of syncope) are detected, EGM data corresponding to the event is stored in memory for subsequent transmission to an external device for physician review. In this regard, the ICM may be configured to continuously record EGM data in a circular buffer. If an arrhythmia or other event of interest is detected, the device saves the EGM data obtained during the arrhythmia or event in memory for later transmission. As such, the memory requirements of the device are relatively modest compared to those of a pacer/ICD, which may record a much larger amount of data. Note that the ICM may also be configured to be activated by the patient using an external device (not shown) whenever the patient feels symptoms indicative of a cardiac ailment, such as heart palpitations, shortness of breath, etc. Even in such implementations, however, it is advantageous to automatically detect arrhythmias, as the patient may not feel symptoms or may be unable to activate the ICM promptly.

As with the pacer/ICD discussed above, to detect supraventricular arrhythmias, the ICM processes the cardiac signals it receives to identify and eliminate irregular ventricular beats (e.g. ectopic beats and bigeminal beats), then analyzes RR intervals to detect AF and other supraventricular arrhythmias using the chaos-based technique discussed below. Therapy is not delivered in response to the arrhythmia since the ICM is not equipped to deliver therapy. Rather, the physician reviews the diagnostic data recorded by the device and, if needed, arranges for a pacer/ICD to be implanted.

Hence, FIG. 3 provides an overview of an ICM capable of, at least, detecting AF and other supraventricular arrhythmias and recording suitable diagnostic information. Embodiments may be implemented that do not necessarily perform all of these functions. For example, embodiments may be implemented that provide only detecting AF but not for detecting other types of arrhythmias or syncope. Note also that the particular location of the implanted device shown in FIG. 3 is merely illustrative and may not necessarily correspond to actual implant location.

### Exemplary Supraventricular Arrhythmia Detection Techniques

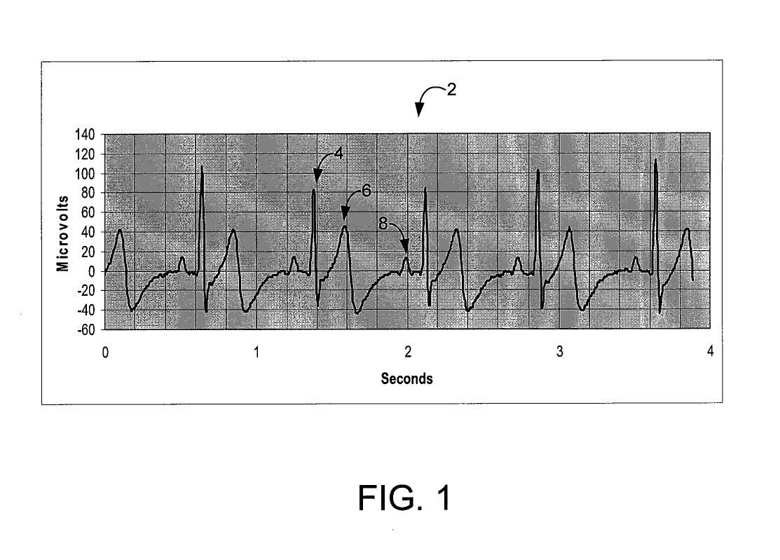

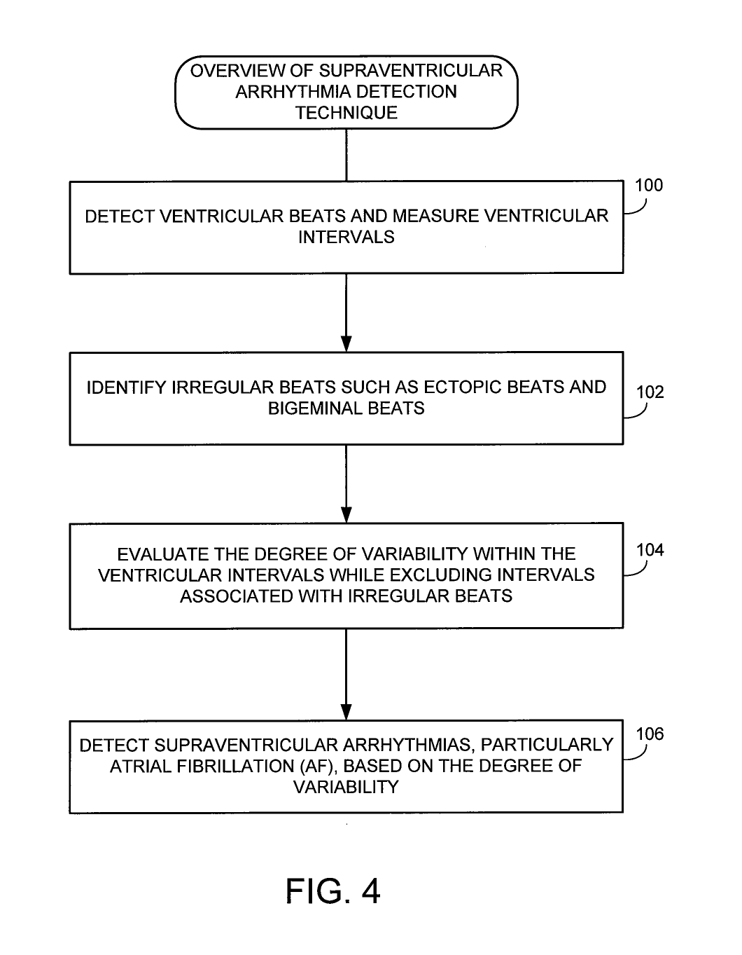

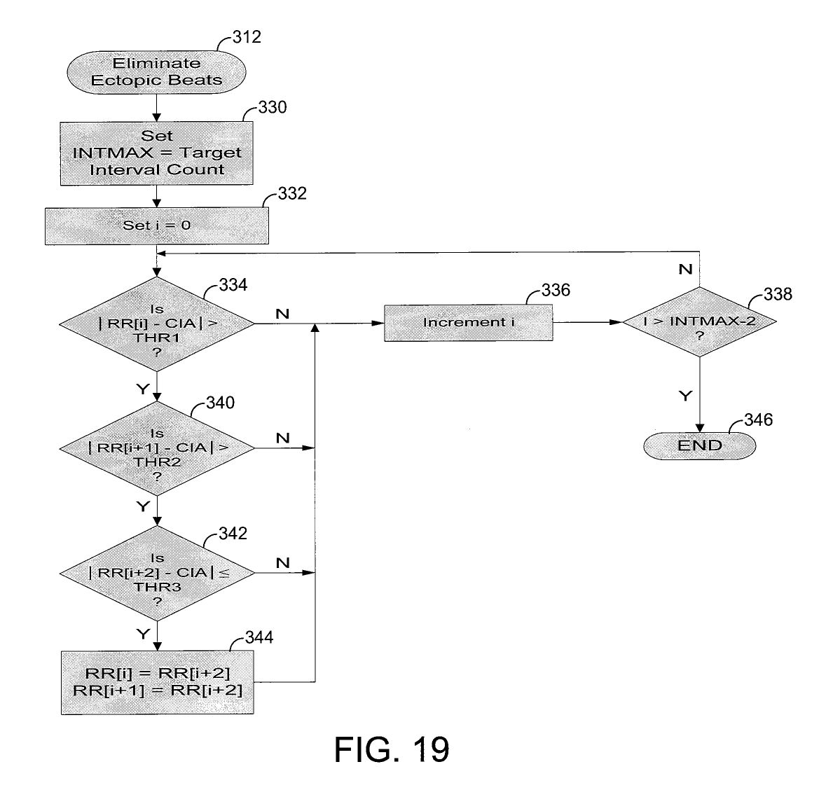

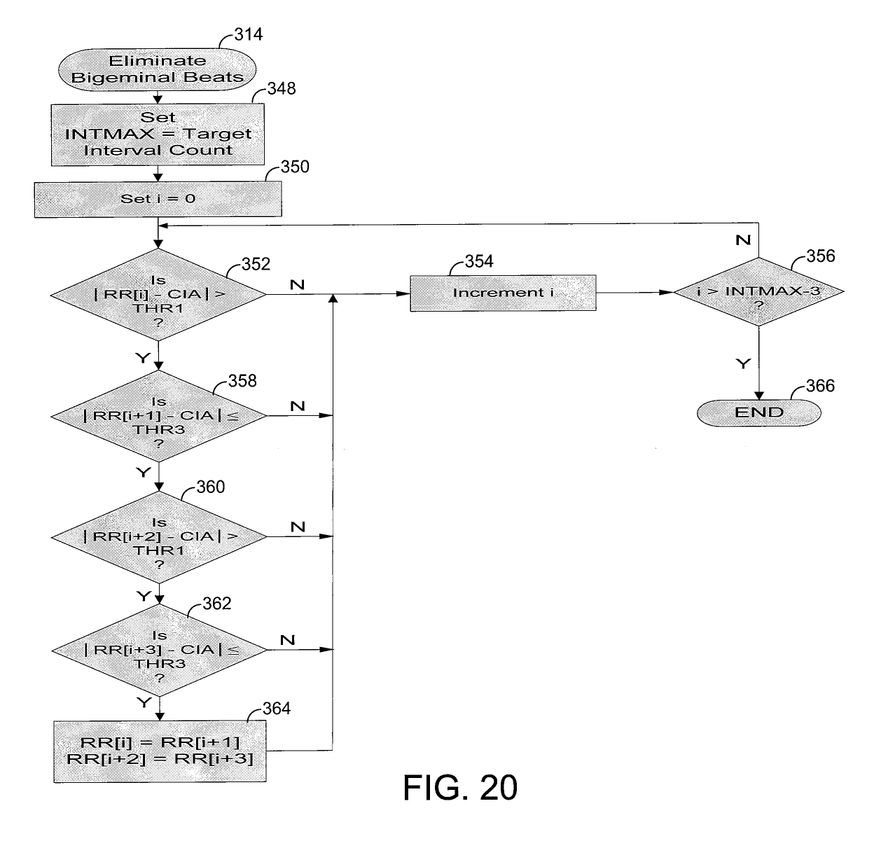

FIG. 4 summarizes a general method for detecting and distinguishing supraventricular arrhythmias within a patient. The method may be performed by the systems of FIG. 1 or 2 or by any other suitably equipped implantable medical system. Briefly, at step 100, the implantable device detects ventricular beats and measures ventricular intervals. Otherwise conventional techniques may be used for detecting the ventricular beats, such as by sensing a ventricular channel IEGM using a pacer/ICD or sensing a subcutaneous EGM using a loop monitor and identifying electrical events having magnitudes exceeding a threshold indicative of ventricular depolarization of the heart. Ventricular intervals may be measure by, e.g., tracking RR intervals. At step 102, the implantable device identifies certain irregular ventricular beats such as ectopic beats and bigeminal beats. Particularly efficient techniques for detecting ectopic beats and bigeminal beats will be discussed below with reference to FIGS. 19-20.

At step 104, the implantable device evaluates the degree of variability within the ventricular intervals while excluding intervals associated with irregular beats. In the various examples described herein, an efficient chaos-based analysis is used to evaluate the degree of variability based dRR intervals, but other suitable techniques may instead be exploited. At step 106, the implantable device then detects supraventricular arrhythmias, particularly AF, based on the degree of variability within the ventricular intervals. Depending upon the implementation, the implantable device can also distinguish among various supraventricular arrhythmias and/or distinguish between supraventricular arrhythmias and ventricular arrhythmias based on the degree of variability within the ventricular intervals.

Hence, supraventricular arrhythmias are detected and distinguished based on the variability of the ventricular intervals, wherein ectopic beats and other irregular beats are eliminated before the variability is calculated, thus mitigating detection problems that might arise if the variability were instead calculated based on all ventricular beats.

Turning now to FIG. 5, pertinent components of an AF detection system 200 for use either in an ICM or in a pacer/ICD are illustrated. Electrical signals are sensed via a sense amplifier 202 and fed into both a relatively wideband filter 204 (having a passband of 0.5-35 Hz) and a relatively narrow band filter 206 (having a passband of 10-35 Hz). Signals output from filter 204 are converted to digital signals via a first analog/digital converter (ADC) 208 for storage within temporary memory (not separately shown) of the implantable device. Signals output from filter 208 are converted to digital signals via a second ADC 210, the fed into an R-wave detector 212 that employs automatic sensitivity control (ASC). See, e.g., U.S. Pat. Nos. 7,155,282, 6,862,476 and 6,539,259.

Signals indicative of R-waves (i.e. ventricular depolarization events) are then routed into an interval extraction unit 214, which determines or extracts the intervals between the R-waves (e.g. RR intervals), while preferably identifying and excluding irregular ventricular beats such as ectopic beats and bigeminal beats. The resulting RR intervals are fed into an AF detector 216, which exploits chaos-based techniques to detect AF, if occurring within the patient. The R-wave detector, the interval extraction unit and the AF detector may be implemented, for example, as embedded firmware within the device. Although not shown in FIG. 5, the AF detector outputs a signal indicative of AF to other components of the implantable device to, e.g., control storage transference of the most recent data stored in the temporary to a more permanent memory (also not separately shown), for subsequent transference to an external system for display and analysis.

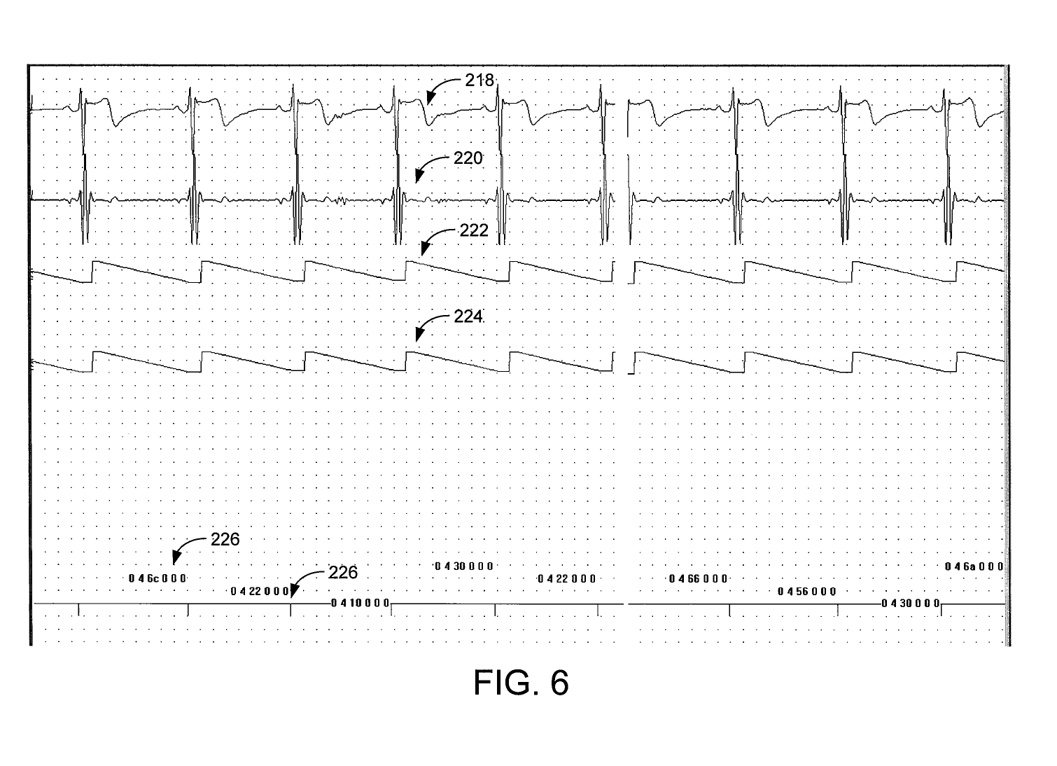

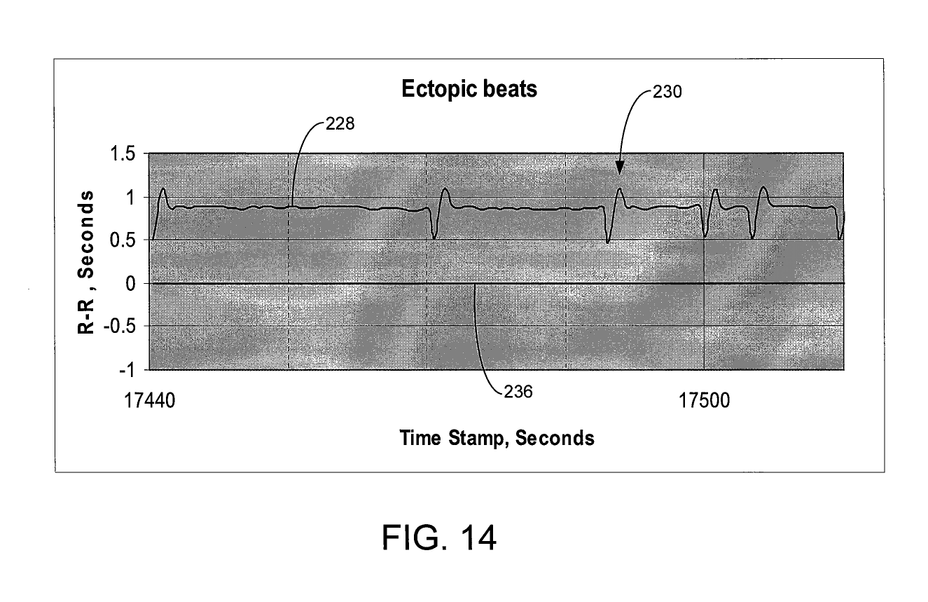

FIG. 6 illustrates exemplary EGM data that may be processed by the components of FIG. 5. Trace 218 is the input signals sensed by the sense amp, with P-waves, QRS-complexes and T-waves visible therein. Trace 220 is the filtered version of the signal output by the narrowband filter, retaining substantially only the QRS-complexes as well as comparatively very low amplitude signals associated with the P-waves and T-waves. Traces 222 and 224 illustrate time-varying detection thresholds employed by ASC components of the R-wave detector. (In other implementations, only a single trace of the time-varying detection thresholds is displayed.) Also shown are markers 226 generated by the firmware of the R-wave detector, which indicate the points/times at which R-waves were detected. In the particular example of FIG. 6, the R-waves are all normal or regular R-waves. It should be understood, however, that even though the refractory period following ventricular sense and the ASC decay delay period are effective in eliminating ectopic R waves, some of the R-waves detected within patient might be ectopic R-waves, bigeminal R-waves, etc. (Techniques for eliminating such irregular ventricular events will be described below beginning with FIG. 14.) Note also that a small gap appears in the various traces of FIG. 6, which is merely indicative of a gap in the data recorded in this example. Such gaps do not appear in the actual data sensed and analyzed by the implantable device, which can sense EGM data substantially continuously.

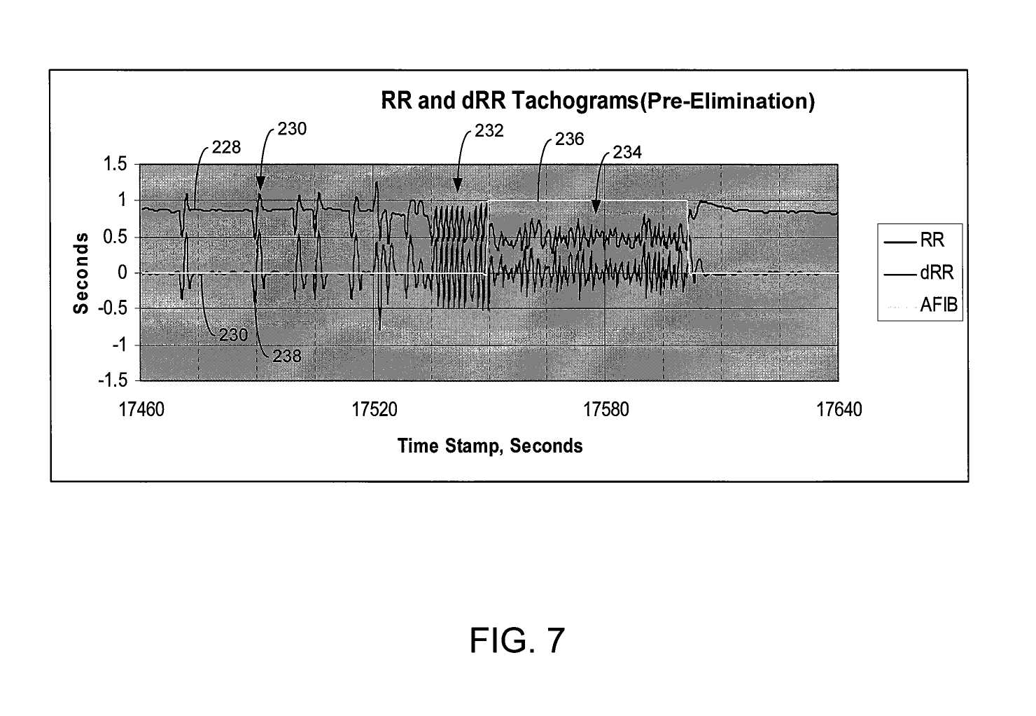

Turning now to FIGS. 7-22, chaos-based techniques for detecting AF will be described in greater detail. FIG. 7 illustrates exemplary tachograms, which highlight changes in RR (or dRR) intervals. More specifically, an RR tachogram 228 is shown, which is representative of changes in RR intervals. So long as the intervals remain constant, the amplitude of the tachogram does not change. Hence, a spike in the RR tachogram, such as spike 230, indicates a temporary change in the RR intervals, likely due to individual irregular ventricular beats, such as ectopic beats. Cyclic variations in the RR tachogram, such as variations 232, indicate cyclical variations in RR intervals, likely due to a series of bigeminal beats. Chaotic variations in RR tachogram, such as variations 234, indicate chaotic variations in RR intervals, likely due to AF. A trace 236 is superimposed over the RR tachogram to identify the location of the episode of AF, where a value of “1” denotes AF and a value of “0” denotes no AF. (AF trace 236 is provided in the figure merely to graphically highlight the episode of AF and is not necessarily generated by the implantable device.)

Similar variations in ventricular intervals are present in a corresponding dRR tachogram 238 of FIG. 7. Note that the value “dRR” is derived from RR by subtracting the most recent RR value from the preceding RR value (i.e. RR[N]−RR[N−1]), in the preferred embodiment. However, in general dRR can be defined with a delay of d, where dRR is defined by subtracting the most recent RR value from the RR value delay d before (i.e. RR[N]−RR[N−d]). The dRR tachogram eliminates the numerical offset appearing in RR tachograms so that uniform RR intervals yield dRR values of zero. Also, the dRR tachogram amplifies changes in RR intervals, as indicated by the larger spikes in trace 238 as compared to trace 228. In reviewing the dRR tachogram of FIG. 7, note that dRR mostly remains near the baseline value of zero due to small but normal RR variations as expected. Sudden spikes represent PACs, PVCs and potentially missed R beats.

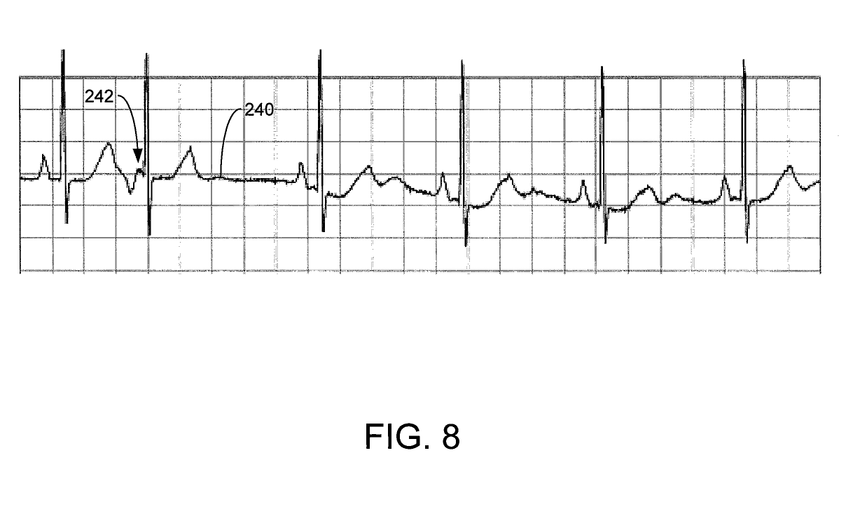

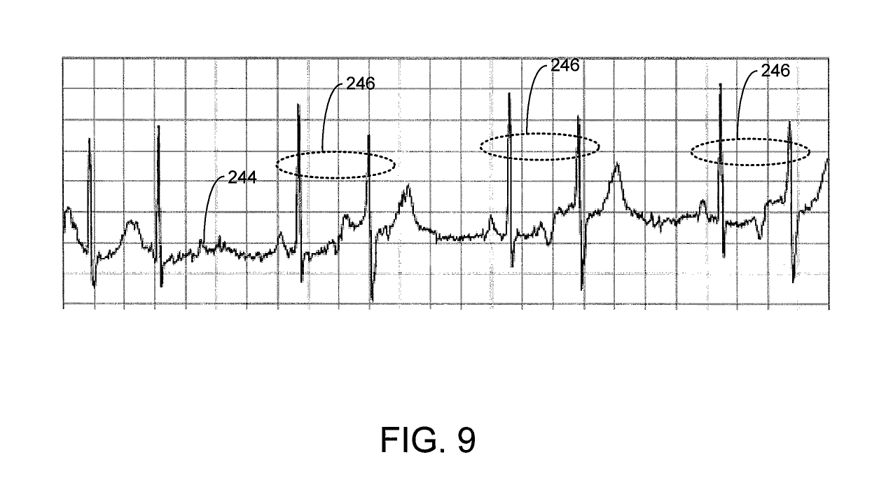

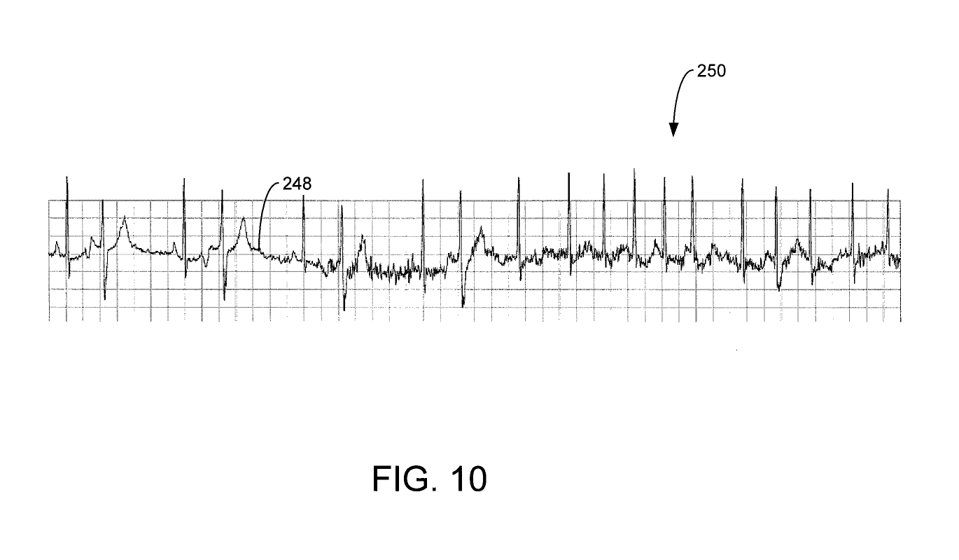

FIG. 8 illustrates a portion 240 of the corresponding EGM with an exemplary PAC 242 identified. (PACs, although atrial events, result in early ventricular depolarization, hence triggering a spike in dRR.) Periodic and oscillatory changes in dRR are indicative of a bigeminal pattern, leading into the AF episode. FIG. 9 illustrates a portion 244 of the corresponding EGM with exemplary bigeminal beats 246 identified. The dRR variations during the AF episode are chaotic and appear random. FIG. 10 illustrates a portion 248 of the corresponding EGM with exemplary AF beats 250 identified. In contrast, atrial flutter (AFL) causes little variation in the ventricular rate and hence little variation in dRR. (Note, though, that AF is not present in the traces of FIGS. 7-10.)

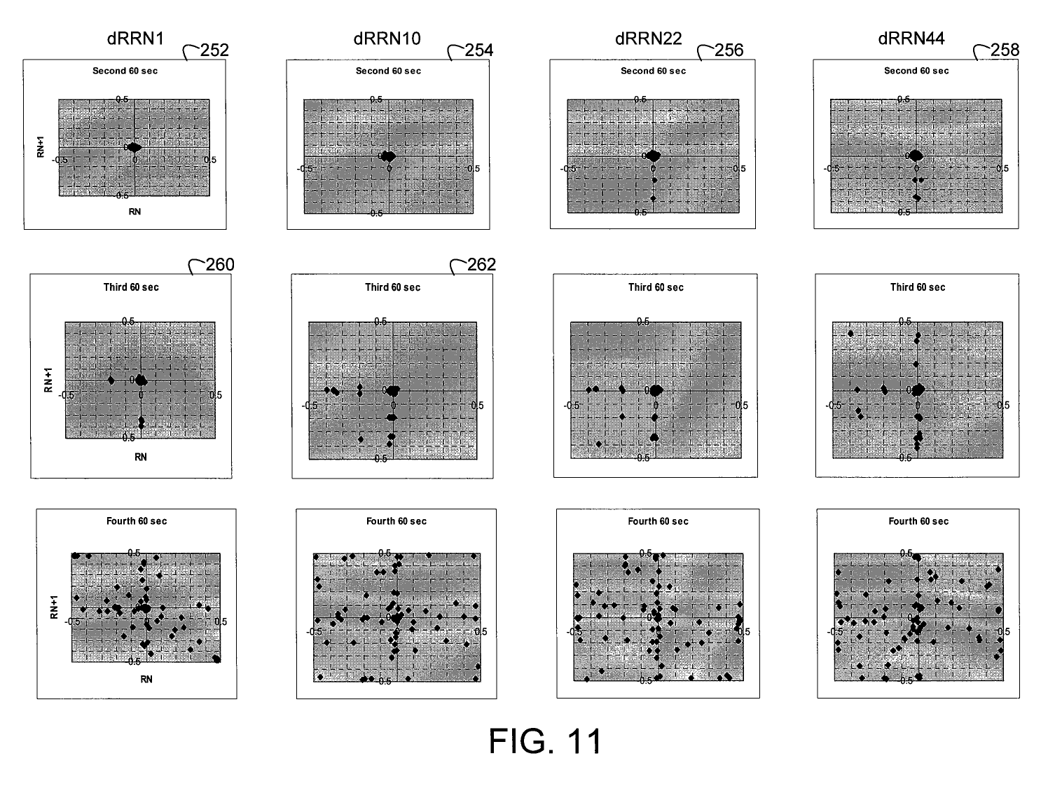

FIGS. 11-13 illustrate various exemplary Poincare plots derived from patient dRR tachograms. Poincare plots may be used to evaluate the degree of variability or chaos within the dRR tachograms so as to detect AF or potentially other supraventricular arrhythmias. FIG. 11 illustrates a series of Poincare plots or maps derived from the dRR tachogram of FIG. 7. The Poincare plots (also referred to as Lorentz plots) were generated by plotting dRR[N−d] with respect to dRR[N], where “d” represents a delay value. More specifically, the exemplary plots of FIG. 11 were plotted for delay values of 1, 10, 22 and 44 (from left to right on each row) and for different 60 second intervals of the tachogram (from top to bottom.)

Herein:

dRRN1 refers to the plot of dRR[N−1] with respect to dRR[N]

dRRN10 refers to the plot of dRR[N−10] with respect to dRR[N]

dRRN22 refers to the plot of dRR[N−22] with respect to dRR[N]

dRRN44 refers to the plot of dRR[N−44] with respect to dRR[N]

Hence, within FIG. 11, plot 252 is the dRRN1 plot derived from data obtained during a second sixty-second interval of data. (No data was plotted for the first sixty-second interval of data.) Plot 254 of the second column is the dRRN10 plot derived from data obtained for the same sixty-second interval of data. Plot 256 is the dRRN22 plot derived from data obtained for the same sixty-second interval of data. Plot 258 is the dRRN44 plot derived from data obtained for the same sixty-second interval of data. Plot 260 of the second row is the dRRN1 plot derived from data obtained during a third sixty-second interval of data. Plot 262 is the dRRN10 plot derived from data obtained for the third sixty-second interval of data. And so on. (Note that these Poincare plots were generated based on EGM data that included irregular ventricular beats. That is, such beats were not identified and eliminated before the dRR intervals were obtained. Alternative techniques will be described below, beginning with FIG. 14, wherein irregular ventricular beats are eliminated before dRR intervals are obtained.)

Individual plots are generated as follows, within dRRN1, the first dRR value to be plotted, e.g. dRR1, is plotted along the X-axis with the Y-axis value set to zero. The next dRR value to be plotted, i.e. dRR2, is then plotted along the Y-axis at the same X-axis value of the previous data point. The next dRR value, dRR3, is then plotted along the X-axis at the same Y-axis value as the previous data point. This process continues until all of the dRR data from the corresponding data set (i.e. the second sixty-second interval of data) is plotted. The same procedure is applied to generate the other Poincare plots using different delay intervals and/or different data sets, as already explained.

As can be seen from the plots of FIG. 11, data within the top row (i.e. data taken from the second sixty-second interval) does not reveal much variability. That is, there is very little scatter or chaos within that data, indicative of uniform RR intervals. The few data points plotted away from the center of the plot are likely due to a few PVCs, PACs, etc. Data within the middle row (i.e. data taken from the third sixty-second interval) shows significantly greater variability, indicative of less uniform RR intervals. The larger number of data points plotted away from the center is likely due to bigeminy. Finally, data within the bottom row (i.e. data taken from the fourth sixty-second interval) shows substantial variability, indicative of chaotic RR intervals. The large number of data points plotted far from the center is likely due to AF.

In other words, as the episode of AF approaches, the amount of scattering or randomness/chaos increases within in the Poincare plots. PVCs, PACs, bigeminal patterns, etc., produce fairly regular patterns within the plots, with minimal data scattered off either the X or Y axes. AF produces much greater scatter in portions of the plots well off either the X or Y axes. Note also that different amounts of delay (i.e. different values for “d”) produce different patterns. Nevertheless, the relative increase in scatter is consistent.

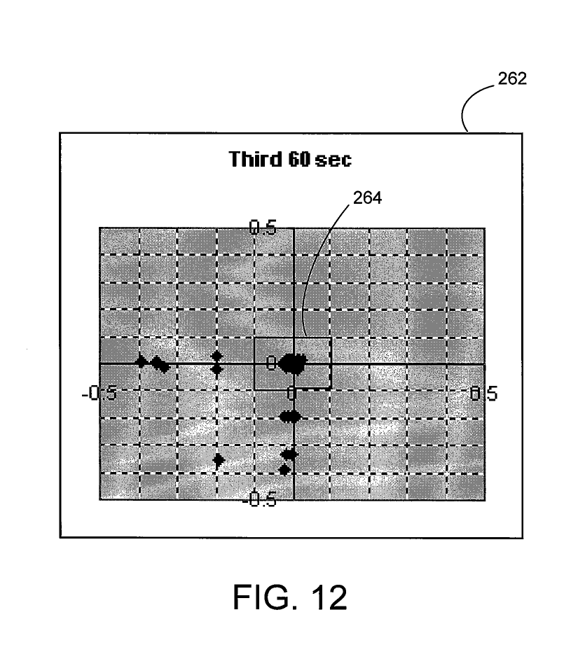

In an exemplary implementation, to take advantage of the scattering characteristics of AF, a threshold of +/−100 msec is used as AF detection criteria on both axes of the X-Y plane. That is, a central box is defined extending to +/−100 msec on the X axis and +/−100 msec on the Y-axis. An example of the center box is shown in the plot of FIG. 12, which corresponds to plot 262 of FIG. 11, but additional illustrates center box 264. Any data points falling outside the box are deemed to be indicative of possible AF and are counted using a memory storage bin. In one example, a separate bin is defined for each value of d. Hence, for the example of FIG. 11, four separate bins are defined corresponding to dRRN1, dRRN10, dRRN22 and dRRN44. The bins are referred to herein as BIN 1, BIN 10, BIN 22, and BIN 44. Each time a data point is detected outside the center box in one of the Poincare plots, the corresponding bin is incremented. At the end of each sixty-second interval or time window, the bin counts are each divided by the total number of data points in the corresponding plot for that time window to thereby calculate a percentage of points outside the center box. At the end of each sixty-second time interval, the bins are cleared. The percentages are recorded for diagnostic purposes and can be examined to detect AF.

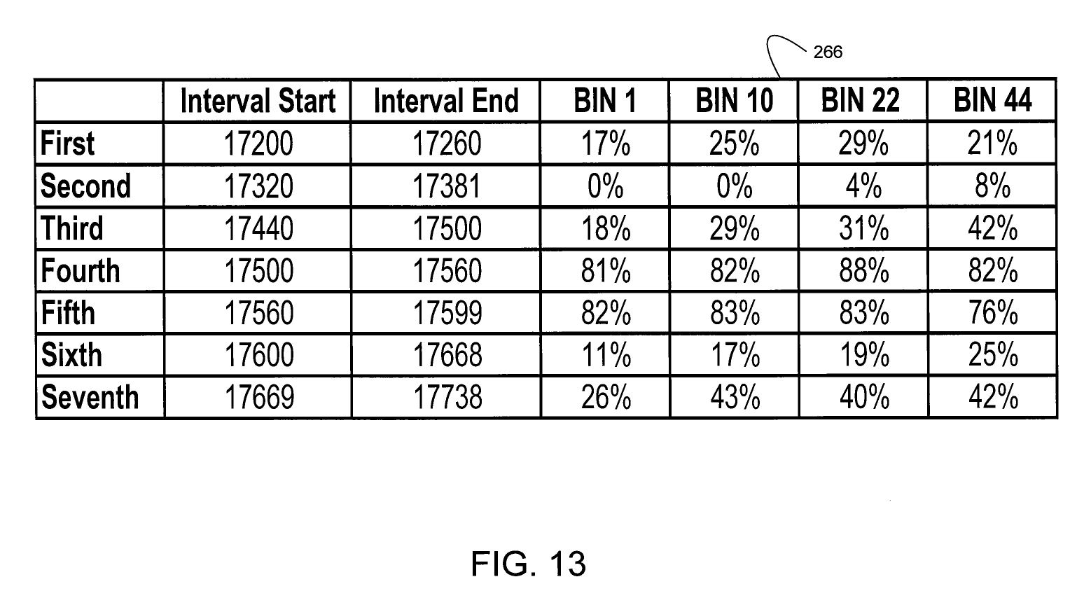

Exemplary percentage data for the various bins is set forth in table 266 of FIG. 13, along with numerical values indicating the start and stop times of the sixty-second intervals. As can be seen, the bin percentages obtained during the fourth and fifth time windows are quite high, each over 75%. These time windows correspond to the AF episode shown in the tachograms of FIG. 7. The high percentages are thus indicative of the episode of AF. Accordingly, the ICM or other implantable device examines the percentages at the completion of each sixty-second interval to detect AF. A variety of specific detection procedures can be used. For example, in one possible implementation, AF is only detected if the percentage for each of the four bins exceeds some predetermined threshold percentage, such as 75%. In another implementation, AF is detected whenever a majority of bins have percentage values exceeding the threshold percentage. Otherwise routine experimentation can be performed to determine optimal threshold comparison strategies and optimal threshold values for use with different implantable systems. Preferably, the threshold values are re-programmable by the clinician, when appropriate.

Hence, FIGS. 11-13 illustrate an exemplary technique for detecting AF using Poincare-type plots based on dRR values. Exemplary graphical plots have been shown and described. It should be understood that the implantable device does not actually plot data in any graphical manner. Rather, the device calculates numerical values representative of the aforementioned Poincare plots and stores the numerical values in memory. The aforementioned bins and tables are also stored numerically in memory. AF detection components of the device (which may be implemented via hardware/software/firmware/etc.) processes the recorded data to detect AF and generate output signals accordingly. As mentioned, within an ICM, AF detector output signals can be used to trigger transference of temporarily stored subcutaneous EGM data into more permanent memory for subsequent clinician review. Within a pacer/ICD, the AF detector output signals may be used to control other device functions, such as to, e.g., confirm detection of AF made using atrial channel signals before cardioversion shocks are delivered.

Moreover, it should be understood that the various detection parameters described above are merely exemplary. For example, other values for the delay interval “d” can be used instead of 10. More or fewer bins can be defined. Different interval durations can also be exploited. That is, intervals other than “sixty-second intervals” can instead be used. The shape and size of the center box described above is also merely exemplary and other boxes may be used having differing sizes and shapes. Otherwise routine experimentation may be performed to determine optimal shapes and sizes for use with different implantable devices.

Still further, routine experimentation may be performed to determine optimal shapes and sizes for use in detecting and distinguishing different types of supraventricular arrhythmias. For example, different boxes may be defined that correspond to the different patterns expected for different arrhythmias (bigeminal patters, trigeminal patterns, PACs, PVCs, AFL, etc.) In this manner, the device can be programmed to easily distinguish among different supraventricular arrhythmias, where appropriate. At least some ventricular arrhythmias may also be detected using the aforementioned techniques. VF, for example, results in significant chaotic variation in RR intervals. Typically, implantable devices can more readily detect ventricular arrhythmias based on a direct analysis of R-waves, particularly the rate of R-waves. Hence, the chaos-based approaches described herein are not necessarily needed for the purpose of detecting ventricular arrhythmias. Nevertheless, within at least some implantable devices and for at least some purposes, it may be advantageous to exploit the techniques describe herein for use in detecting and distinguishing ventricular arrhythmias, as well as supraventricular arrhythmias. See, U.S. Pat. No. 5,439,004 of Duong-Van, et al., entitled “Device and Method for Chaos Based Cardiac Fibrillation Detection.”

As noted, the Poincare plots of FIGS. 11-13 were generated without first eliminating irregular ventricular beats, such as bigeminal beats. With reference to FIGS. 14-22, AF detection techniques will be described wherein irregular ventricular beats are identified and eliminated before Poincare plots are generated. By eliminating irregular ventricular beats first, AF detection specificity is improved, since any scatter due to the irregular beats will not appear within the dRR Poincare plots. Also, the following techniques provide procedures for distinguishing sinus tachycardia from AF, as both can result in substantial scatter within the dRR Poincare plots.

Returning briefly to FIG. 7, the tachograms shown therein include isolated spikes 230 due to ectopic beats, cyclical variations 232 due to bigeminal beats, and chaotic variations 234 due to AF. The spikes due to ectopic beats with the RR tachogram are shown more clearly in FIG. 14. In this expanded view, which shows only the RR tachogram, it is possible to see the clear signature of the ectopic beat. Each ectopic beat starts with a negative deflection from the baseline rhythm, which is greater than 150 msec, followed by a positive deflection above the baseline >100 msec, due to the compensatory pause and finally back to the baseline. (The PAC causing the ectopic beat is shown in the EGM trace of FIG. 8, described above.) Note that FIG. 14 also includes trace 236, which again represents an AF detection trace. Trace 236 remains at zero throughout FIG. 14, since AF is not yet present.

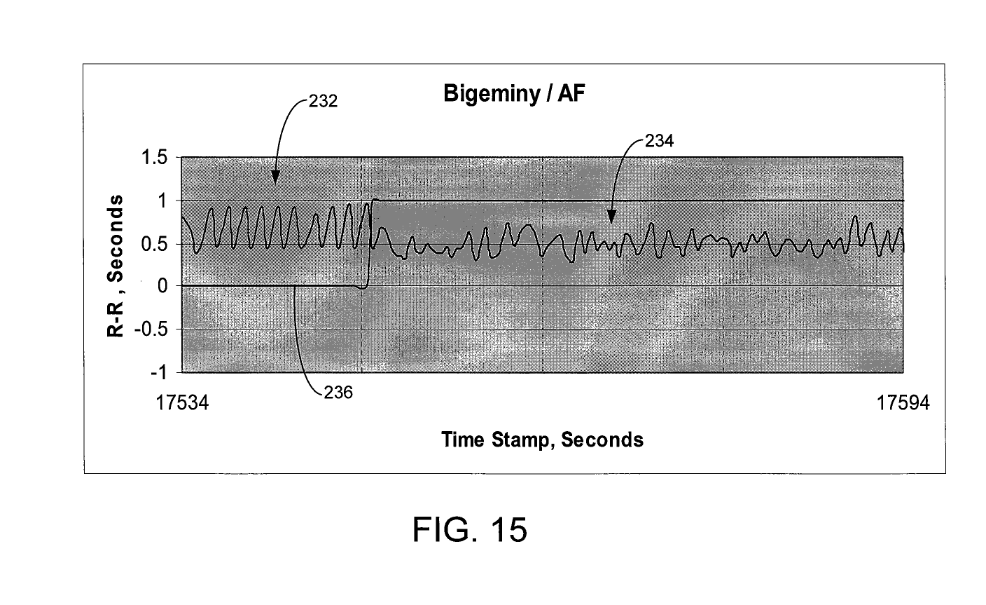

The cyclical variations due to bigeminal beats leading to AF are shown more clearly in FIG. 15. In the expanded view, it is possible to see the clear signature of the bigeminal beats, which is a repeating pattern of long-short intervals, where the long interval is a value close to the baseline variations of the rhythm. (The bigeminal beats themselves are shown in the EGM trace of FIG. 9, described above.)

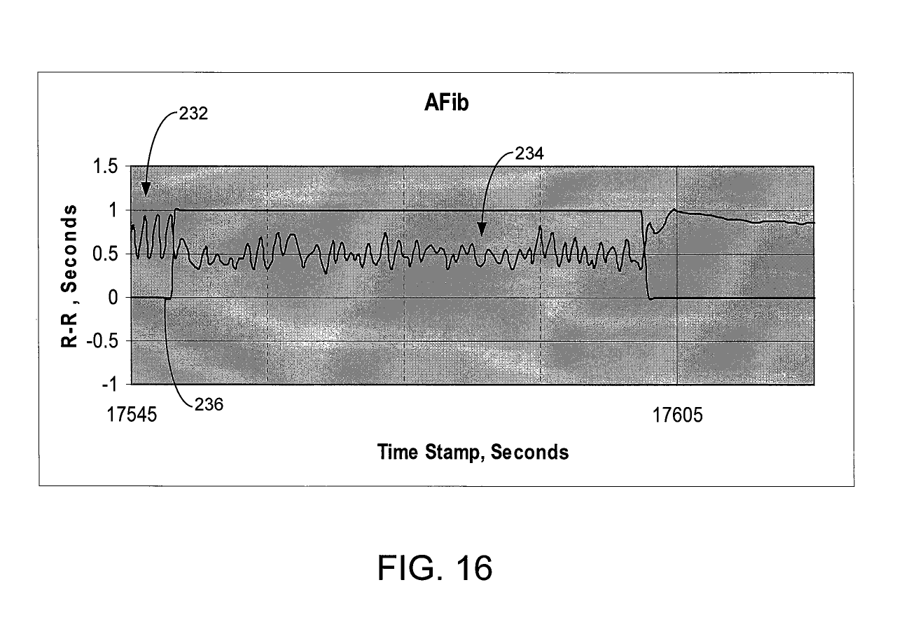

The episode of AF is shown more clearly in FIG. 16, its duration marked by the AF detection trace 236, which has a value of one during the episode. In the expanded view, it is possible to see the clear signature of the chaotic RR tachogram in response to atrial fibrillation. The signature is chaotic due to almost random variations in the ventricular intervals. (The ventricular beats themselves are shown in the EGM trace of FIG. 10, described above.)

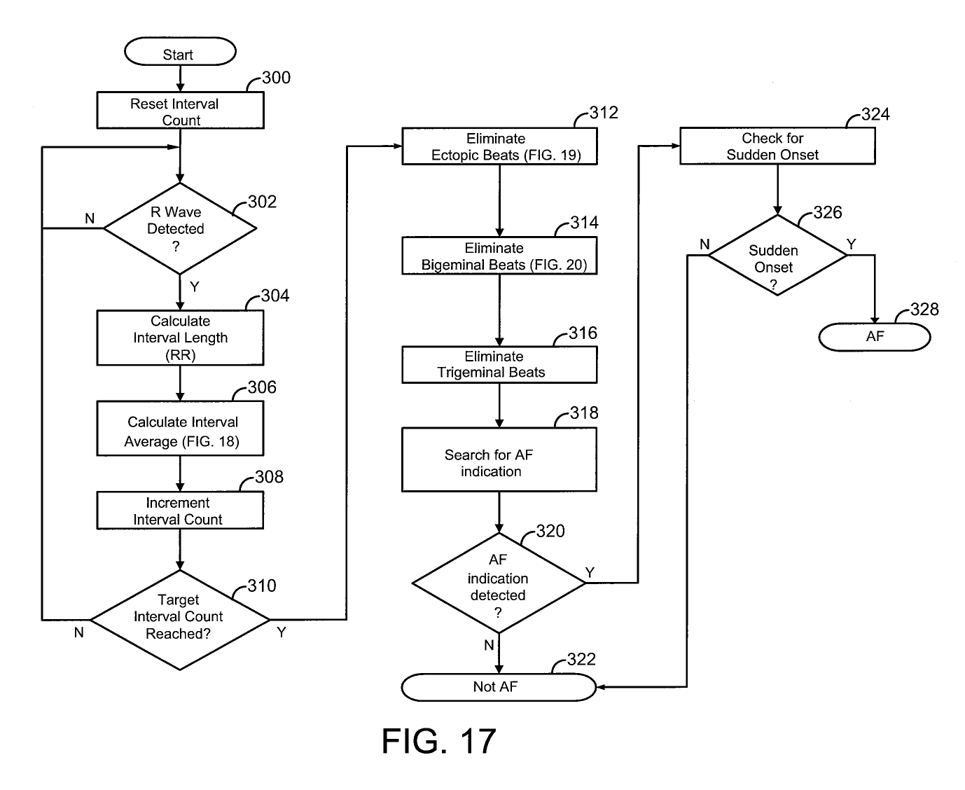

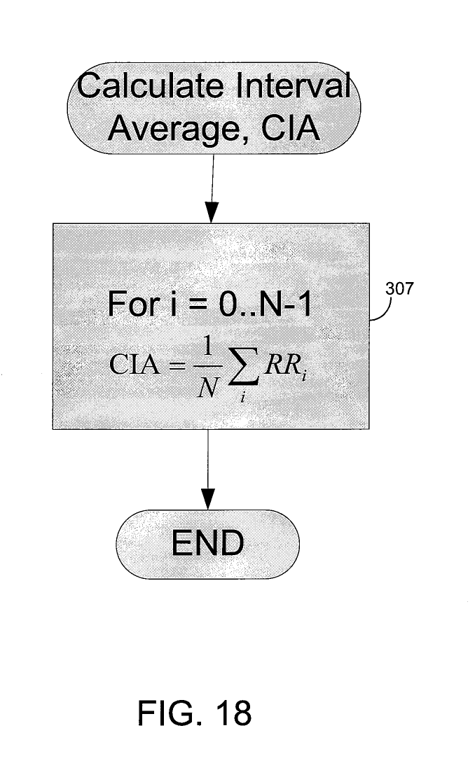

FIGS. 17-19 illustrate a technique for detecting AF wherein irregular ventricular beats are eliminated prior to AF detection. FIG. 17 provides an overview of the technique. At step 300, the implantable device resets an interval counter used to count the RR intervals as new R-waves are detected. Whenever a new R-wave is detected, at step 302, the device calculates the length of the RR interval, at step 304, and calculates the latest average value for the intervals. The average is based, e.g. only on those intervals detected and counted since the last reset of the interval counter. FIG. 18 illustrates the calculation of the interval average, which is referred to herein as the calculated interval average (CIA). The average is calculated, at step 307, using the individual RR intervals and the current value of the counter. The average is used later in the detection and elimination of ectopic beats and other irregular ventricular beats.

Returning to FIG. 17, at step 308, the interval count is incremented and, so long as the count as not yet reached a predetermined target value, as determined at step 310, processing returns to step 302 to detect additional R-waves. In one example, the target count is set in the range of sixty to eighty so that about one minute's worth of interval data is collected before the intervals are then processed to eliminate irregular beats and detect AF. Alternative, rather than counting the number of R-waves, the device could instead use a timer to track sixty-second time windows, as described above in connection with FIGS. 11-13.

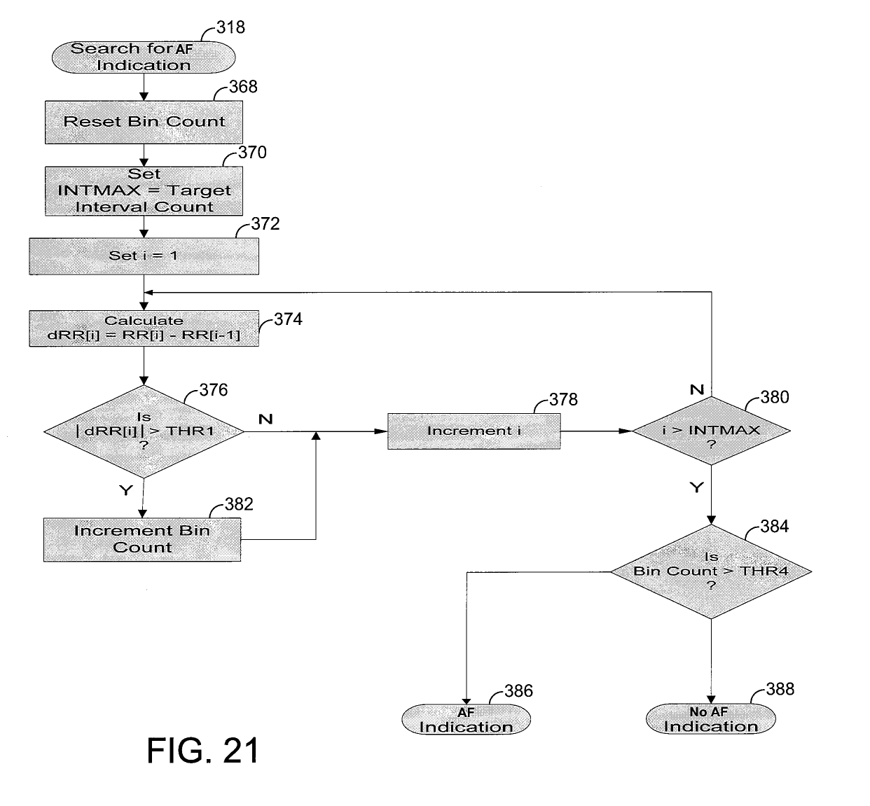

Once the target count has been reached (or the timer has elapsed), processing then proceeds to step 312 where the implantable device detects and eliminates ectopic beats, which will be described below with reference to FIG. 20. Next, at step 314, the device detects and eliminates bigeminal beats, which will be described below with reference to FIG. 21. Then, at step 316, the device detects and eliminates trigeminal beats using similar techniques. Note that the elimination of trigeminal beats is optional in the example of FIG. 17. Also, note that additional eliminating steps could be performed, such as to eliminate quadrigeminal beats. At step 318, the device then searches for AF using techniques to be described below with reference to FIG. 21. Assuming AF is not detected then, following step 320, an output signal indicative of no AF is generated. Although not specifically shown, the implantable device then preferably returns to step 300 to restart the procedure to examine the next group of RR intervals.

However, if an episode of AF is indicated, then step 324 is performed where the implantable device determines whether the episode occurred relatively suddenly, i.e. the device checks for sudden onset. Sudden onset may be detected, for example, by examining changes in the bin percentage values from one time window to the next. As described above, high percentages are indicative of AF or ST. A sharp increase in the percentages from previous levels indicates a sudden onset, which is indicative of AF. A more gradual increase is more indicative of ST. If sudden onset is detected, at step 326, the episode is therefore deemed to be AF and suitable output signals are generated, at step 328. Otherwise, the episode is deemed to be sinus tachycardia (or some other non-AF episode), and processing returns to step 322. In either case, although not specifically shown, processing returns to step 300 to initiate another AF detection cycle.

Turning now to FIG. 19, an exemplary technique for detecting and eliminating ectopic beats is shown for use at step 312 of FIG. 17. Beginning at step 330, the implantable device sets an interval max (INTMAX) value equal to the target interval count, then sets an internal loop counter i to 0, at step 332. Beginning at step 334, the device then performs a series of calculations using the previously determined CIA value to detect and eliminate ectopic beats. That is, at step 334, the device calculates |RR[i]−CIA| and compares that value against a first threshold (THR1). RR[i] is the RR interval value for the current loop counter value and so the resulting value represents the difference between the current RR value and the average RR value.

If this first difference value does exceed the first threshold, i.e. RR[i] is more or less average, then RR[i] is not an ectopic beat and processing proceeds to step 336 to increment the i counter and then to step 338 to determine if RR[i] represented the last of the RR intervals to be examined. Assuming, though, that the first difference value exceeded the first threshold, i.e. RR[i] was either much larger than or much smaller than the average RR value, then RR[i] might be an ectopic beat and hence is a possible candidate for elimination. However, further tests are required to determined whether RR[i] is indeed an ectopic beats and so step 340 is performed where the device calculates |RR[i+1]−CIA| and compares that value against a second threshold (THR2) where RR[i+1] is the next RR interval value. If this second difference value does not exceed the second threshold, i.e. RR[i+1] is also more or less average, then RR[i] was not an ectopic beat and processing again proceeds to step 336 to increment i. In this regard, ectopic beats tend to be isolated and hence affect both the immediately preceding and the immediately succeeding RR intervals. Hence, both RR[i] and RR[i+1] should differ significantly from the average RR interval. Unless both RR[i] and RR[i+1] significantly differ from the average RR interval value (i.e. CIA), the current ventricular beat is not ectopic.

Assuming, though, that the second difference value exceeded the second threshold, then one additional test is performed before RR[i] is deemed to be ectopic and is eliminated. That is, at step 342, the device calculates |RR[i+2]−CIA| and compares that value against a third threshold (THR3) where RR[i+2] is the next subsequent RR interval value. If this third difference value exceeds the third threshold, i.e. RR[i+2] is also either much larger than or much smaller than the average RR value, then RR[i] is again deemed not to be an ectopic beat and processing proceeds to step 336 to increment i. That is, if RR[i], RR[i+1] and RR[i+2] are all aberrant, then RR[i] is probably not an individual ectopic beat but is instead the first beat in a pattern of aberrant beats. Assuming, though, that the third difference value was less than or equal to the third threshold, i.e. RR[i+2] was more or less average, then RR[i] is deemed to be ectopic since only RR[i] and RR[i+1] were aberrant. In this context, RR[i] represents the ectopic, RR[i+1] the compensatory pause and RR[i+2] the following regular interval. Accordingly, step 340 is performed where the implantable device eliminates RR[i] by resetting RR[i]=RR[i+2] and also resetting RR[i+1]=RR[i+2]. That is, both RR[i] and RR[i+1] are reset to comparatively normal values so they will not adversely affect subsequent AF detection.

Steps 334-344 are repeated until all of the RR intervals in the current set of intervals (i.e. within the current time window) have been analyzed and any and all ectopic beats have been eliminated therein. Following END step 346, processing returns to FIG. 17 for further processing. Note that the threshold values THR1, THR2 and THR3 are predetermined and may be expressed, e.g., as a percentage of the average RR value. For example, the thresholds might be set so as to detect any RR intervals that differ from the average RR value by more than, e.g., 10% to 50%. Otherwise routine experimentation may be performed to determine optimal values. Also, note that THR1, THR2 and THR3 need not be different and can instead all be set to the same value.

FIG. 20 illustrates an exemplary technique for detecting and eliminating bigeminal beats for use at step 314 of FIG. 17. The general strategy of the technique of FIG. 20 for eliminating bigeminal beats is similar to the technique of FIG. 19 for eliminating ectopic beats and hence only pertinent difference will be described in detail. As before, at step 348, the implantable device sets INTMAX equal to the target interval count, then sets an i to 0, at step 350. Beginning at step 352, the device then performs a series of calculations using the CIA value to detect and eliminate bigeminal beats. At step 352, the device determines whether |RR[i]−CIA exceeds the first threshold (THR1) and, if not, then RR[i] is not a bigeminal beat and so processing proceeds to step 354 to increment the i counter and then to step 356 to determine if RR[i] represented the last of the RR intervals to be examined.

Assuming, though, that the first difference value exceeded the first threshold, then step 358 is performed where the device determines whether |RR[i+1]−CIA| exceeds the third threshold (THR3) and, if so, then RR[i] is not a bigeminal beat and processing again proceeds to step 354. In this regard, bigeminal beats tend to alternate and hence, if RR[i] is bigeminal, then RR[i+1] should not also differ significantly from the average RR interval. That is, if both RR[i] and RR[i+1] are aberrant, then RR[i] is not bigeminal. Assuming, though, that the RR[i] is still a candidate bigeminal beat, then step 360 is performed where the device compares |RR[i+2]−CIA| to the first threshold (THR1). If RR[i+2] is not also aberrant, then RR[i] is not bigeminal. Again, bigeminal beats tend to alternate and so RR[i] and RR[i+2] should not both differ significantly from the average RR interval. Assuming that the RR[i] is still a candidate bigeminal beat, then step 362 is performed where the device compares |RR[i+3]−CIA| to the third threshold (THR3). If this final difference value does not exceed the third threshold, then RR[i] is deemed to be bigeminal since only RR[i] and RR[i+2] were aberrant but RR[i+1] and RR[i+3] were not. Accordingly, step 364 is performed where the implantable device eliminates RR[i] by resetting RR[i]=RR[i+2] and also eliminates RR[i+2] by resetting RR[i+2]=RR[i+3]. That is, both RR[i] and RR[i+2] are reset to comparatively normal values so they will not adversely affect subsequent AF detection.

Steps 352-356 are repeated until all of the RR intervals in the current set of intervals (i.e. within the current time window) have been analyzed and any and all bigeminal beats have been eliminated therein. Following step 366, processing returns to FIG. 17 for further processing. Note that the threshold values THR1, THR2 and THR3 may be the same values as used in FIG. 19 or may potentially be set to different values. Similar logic as in FIGS. 19 and 20 may be used to detect and eliminate trigeminal beats, quadrigeminal beats, etc.

FIG. 21 illustrates an exemplary technique for detecting an indication of AF for use at step 318 of FIG. 17. At step 368, the implantable device resets a bin count to zero, which will be used to count values within a single bin. (Bins are described above with reference to FIG. 13.) At step 370, the device then sets INTMAX equal to the target interval count and, at step 372, sets i to 0. Beginning at step 374, the device then performs a series of dRR calculations to determine whether AF is indicated. That is, at step 374, the device calculates dRR[i] from RR[i] and RR[i+1]. As a reminder, ectopic and bigeminal RR intervals have already been eliminated so that any remaining aberrant intervals are likely due to AF or ST. At step 376, the device compares |dRR[i]| to the first threshold (THR1). If |dRR[i]| does not exceed the threshold, i.e., dRR is more or less normal, then dRR[i] is not indicative of AF and so i is incremented, at step 378, then i is compared against INTMAX to determine if RR[i] represented the last of the RR intervals to be examined.

Assuming, though, that |dRR[i]| exceeded the threshold, then step 382 is performed where the device increments the bin counter. That is, if |dRR[i]| exceeds the threshold, the dRR value is deemed to be outside the aforementioned box (see box 264 of FIG. 12) and hence is indicative of possible AF. Accordingly, the bin is incremented. Note that, in the example of FIG. 21, only a single bin is used. As explained above, multiple bins may instead be exploited. In any case, once the bin is incremented, i is also incremented, at step 378. Once i exceeds INTMAX, step 358 is performed where the device determined whether the bin count exceeds an AF indication threshold (THR4), at step 384. If so, AF is indicated at step 386. Otherwise, AF is not indicated. Alternatively, rather than compare the bin count directly to a numerical threshold, the bin count can be re-expressed as a percentage and compared against a percentage threshold, as discussed above. In any case, if AF is indicated, processing returns to FIG. 17 where the device determines whether AF is in fact present based on the sudden onset of the arrhythmia.

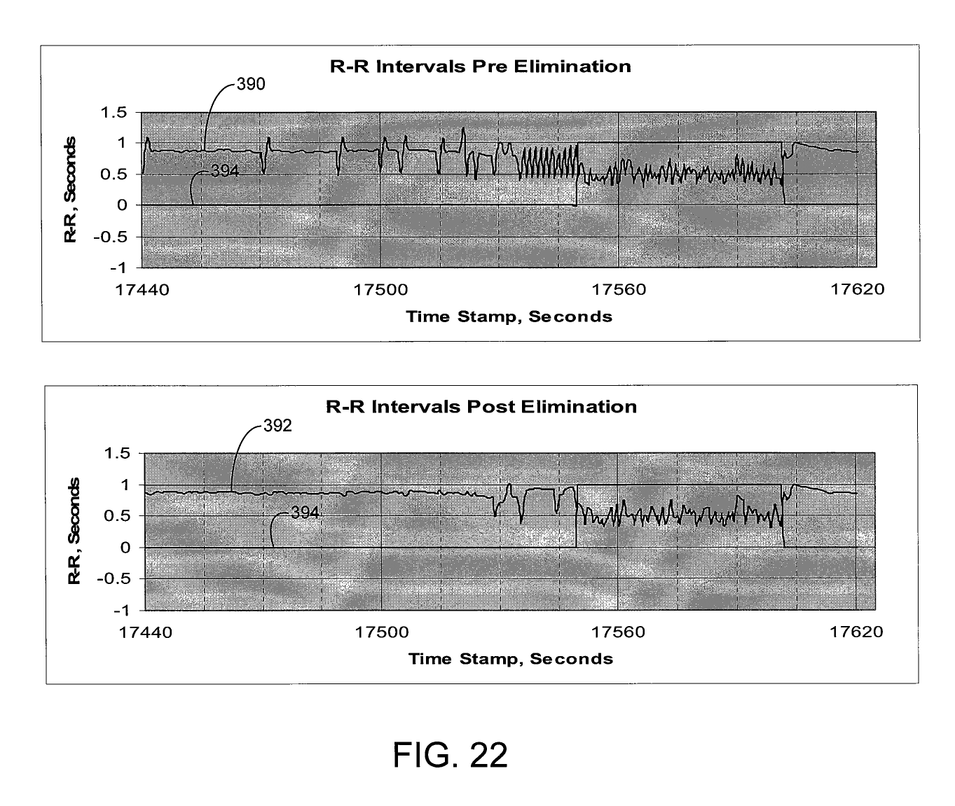

Thus, FIGS. 17-21 set forth a technique for detecting AF wherein ectopic beats, bigeminal beats and potentially other irregular ventricular beats are identified and eliminated. FIG. 22 illustrates the efficacy of this strategy. RR tachogram 390 is a pre-elimination tachogram wherein ectopic beats and bigeminal beats significantly affect the tachogram. RR tachogram 392 is a post-elimination tachogram wherein ectopic beats and bigeminal beats have been substantially filtered out. Nevertheless, the filtering does not significantly affect the chaotic nature of the tachogram during the episode of AF, which is marked via trace 394. Hence, AF can still be detected using chaos-based approaches, without any significant risk that ectopic beats, bigeminal beats, etc., might trigger a false detection of AF. That is, filtering of ectopic and junctional beats thereby tends to improve the specificity by which AF is detected. It should be noted that some noise still appears within the post-elimination tachogram. In this regard, R-wave detection errors due to over/under sensing caused by noise or other factors can result in positive/negative deflections around a baseline rhythm. The elimination techniques described above may not remove all such these artifacts. However, such isolated events will not significantly degrade the specificity of AF detection, which is based on RR intervals collected over time windows of about sixty-seconds.

What have been described are various techniques for detecting AF, which may be exploited with a pacer/ICD, ICM, or other implantable medical device. For the sake of completeness, a detailed description of an exemplary pacer/ICD will now be provided. However, principles of invention may be implemented within other pacer/ICD implementations or within other implantable medical devices. As already explained, pacer/ICDs equipped with an atrial lead will usually examine atrial channel signals to detect AF. However, the chaos-based techniques describe above which exploit ventricular signals might be employed within a pacer/ICD to, e.g., corroborate any AF detection made via atrial channel signals before cardioversion shocks are delivered, or for other purposes.

### Exemplary Pacer/ICD

With reference to FIGS. 23 and 24, a description of an exemplary pacer/ICD will now be provided. FIG. 23 provides a simplified block diagram of the pacer/ICD, which is a dual-chamber stimulation device capable of treating both fast and slow arrhythmias with stimulation therapy, including cardioversion, defibrillation, and pacing stimulation. To provide atrial chamber pacing stimulation and sensing, pacer/ICD 410 is shown in electrical communication with a heart 412 by way of a left atrial lead 420 having an atrial tip electrode 422 and an atrial ring electrode 423 implanted in the atrial appendage. Pacer/ICD 410 is also in electrical communication with the heart by way of a right ventricular lead 430 having, in this embodiment, a ventricular tip electrode 432, a right ventricular ring electrode 434, a right ventricular (RV) coil electrode 436, and a superior vena cava (SVC) coil electrode 438. Typically, the right ventricular lead 430 is transvenously inserted into the heart so as to place the RV coil electrode 436 in the right ventricular apex, and the SVC coil electrode 438 in the superior vena cava. Accordingly, the right ventricular lead is capable of receiving cardiac signals, and delivering stimulation in the form of pacing and shock therapy to the right ventricle.

To sense left atrial and ventricular cardiac signals and to provide left chamber pacing therapy, pacer/ICD 410 is coupled to a CS lead 424 designed for placement in the “CS region” via the CS os for positioning a distal electrode adjacent to the left ventricle and/or additional electrode(s) adjacent to the left atrium. As used herein, the phrase “CS region” refers to the venous vasculature of the left ventricle, including any portion of the CS, great cardiac vein, left marginal vein, left posterior ventricular vein, middle cardiac vein, and/or small cardiac vein or any other cardiac vein accessible by the CS. Accordingly, an exemplary CS lead 424 is designed to receive atrial and ventricular cardiac signals and to deliver left ventricular pacing therapy using at least a left ventricular tip electrode 426, left atrial pacing therapy using at least a left atrial ring electrode 427, and shocking therapy using at least a left atrial coil electrode 428. With this configuration, biventricular pacing can be performed. Although only three leads are shown in FIG. 6, it should also be understood that additional stimulation leads (with one or more pacing, sensing and/or shocking electrodes) might be used in order to efficiently and effectively provide pacing stimulation to the left side of the heart or atrial cardioversion and/or defibrillation.

A simplified block diagram of internal components of pacer/ICD 410 is shown in FIG. 24. While a particular pacer/ICD is shown, this is for illustration purposes only, and one of skill in the art could readily duplicate, eliminate or disable the appropriate circuitry in any desired combination to provide a device capable of treating the appropriate chamber(s) with cardioversion, defibrillation and pacing stimulation as well as providing for the aforementioned AF detection.

The housing 440 for pacer/ICD 410, shown schematically in FIG. 24, is often referred to as the “can”, “case” or “case electrode” and may be programmably selected to act as the return electrode for all “unipolar” modes. The housing 440 may further be used as a return electrode alone or in combination with one or more of the coil electrodes, 428, 436 and 438, for shocking purposes. The housing 440 further includes a connector (not shown) having a plurality of terminals, 442, 443, 444, 446, 448, 452, 454, 456 and 458 (shown schematically and, for convenience, the names of the electrodes to which they are connected are shown next to the terminals). As such, to achieve right atrial sensing and pacing, the connector includes at least a right atrial tip terminal (A_{R} TIP) 442 adapted for connection to the atrial tip electrode 422 and a right atrial ring (A_{R} RING) electrode 443 adapted for connection to right atrial ring electrode 423. To achieve left chamber sensing, pacing and shocking, the connector includes at least a left ventricular tip terminal (V_{L} TIP) 444, a left atrial ring terminal (A_{L} RING) 446, and a left atrial shocking terminal (A_{L} COIL) 448, which are adapted for connection to the left ventricular ring electrode 426, the left atrial ring electrode 427, and the left atrial coil electrode 428, respectively. To support right chamber sensing, pacing and shocking, the connector further includes a right ventricular tip terminal (V_{R} TIP) 452, a right ventricular ring terminal (V_{R} RING) 454, a right ventricular shocking terminal (V_{R} COIL) 456, and an SVC shocking terminal (SVC COIL) 458, which are adapted for connection to the right ventricular tip electrode 432, right ventricular ring electrode 434, the V_{R} coil electrode 436, and the SVC coil electrode 438, respectively.

At the core of pacer/ICD 410 is a programmable microcontroller 460, which controls the various modes of stimulation therapy. As is well known in the art, the microcontroller 460 (also referred to herein as a control unit) typically includes a microprocessor, or equivalent control circuitry, designed specifically for controlling the delivery of stimulation therapy and may further include RAM or ROM memory, logic and timing circuitry, state machine circuitry, and I/O circuitry. Typically, the microcontroller 460 includes the ability to process or monitor input signals (data) as controlled by a program code stored in a designated block of memory. The details of the design and operation of the microcontroller 460 are not critical to the invention. Rather, any suitable microcontroller 460 may be used that carries out the functions described herein. The use of microprocessor-based control circuits for performing timing and data analysis functions are well known in the art.

As shown in FIG. 24, an atrial pulse generator 470 and a ventricular pulse generator 472 generate pacing stimulation pulses for delivery by the right atrial lead 420, the right ventricular lead 430, and/or the CS lead 424 via an electrode configuration switch 474. It is understood that in order to provide stimulation therapy in each of the four chambers of the heart, the atrial and ventricular pulse generators, 470 and 472, may include dedicated, independent pulse generators, multiplexed pulse generators or shared pulse generators. The pulse generators, 470 and 472, are controlled by the microcontroller 460 via appropriate control signals, 476 and 478, respectively, to trigger or inhibit the stimulation pulses.

The microcontroller 460 further includes timing control circuitry (not separately shown) used to control the timing of such stimulation pulses (e.g., pacing rate, AV delay, atrial interconduction (inter-atrial) delay, or ventricular interconduction (V-V) delay, etc.) as well as to keep track of the timing of refractory periods, blanking intervals, noise detection windows, evoked response windows, alert intervals, marker channel timing, etc., which is well known in the art. Switch 474 includes a plurality of switches for connecting the desired electrodes to the appropriate I/O circuits, thereby providing complete electrode programmability. Accordingly, the switch 474, in response to a control signal 480 from the microcontroller 460, determines the polarity of the stimulation pulses (e.g., unipolar, bipolar, combipolar, etc.) by selectively closing the appropriate combination of switches (not shown) as is known in the art. In addition, the switch includes components for selectively coupling the atrial tip and ring electrodes in parallel during an AF risk assessment procedure.

Atrial sensing circuits 482 and ventricular sensing circuits 484 may also be selectively coupled to the right atrial lead 420, CS lead 424, and the right ventricular lead 430, through the switch 474 for detecting the presence of cardiac activity in each of the four chambers of the heart. Accordingly, the atrial (ATR. SENSE) and ventricular (VTR. SENSE) sensing circuits, 482 and 484, may include dedicated sense amplifiers, multiplexed amplifiers or shared amplifiers. The switch 474 determines the “sensing polarity” of the cardiac signal by selectively closing the appropriate switches, as is also known in the art. In this way, the clinician may program the sensing polarity independent of the stimulation polarity. Each sensing circuit, 482 and 484, preferably employs one or more low power, precision amplifiers with programmable gain and/or automatic gain control and/or automatic sensitivity control, bandpass filtering, and a threshold detection circuit, as known in the art, to selectively sense the cardiac signal of interest. The automatic gain/sensitivity control enables pacer/ICD 410 to deal effectively with the difficult problem of sensing the low amplitude signal characteristics of atrial or ventricular fibrillation. The outputs of the atrial and ventricular sensing circuits, 482 and 484, are connected to the microcontroller 460 which, in turn, are able to trigger or inhibit the atrial and ventricular pulse generators, 470 and 472, respectively, in a demand fashion in response to the absence or presence of cardiac activity in the appropriate chambers of the heart.

For arrhythmia detection, pacer/ICD 410 utilizes the atrial and ventricular sensing circuits, 482 and 484, to sense cardiac signals to determine whether a rhythm is physiologic or pathologic. As used herein “sensing” is reserved for the noting of an electrical signal, and “detection” is the processing of these sensed signals and noting the presence of an arrhythmia. The timing intervals between sensed events (e.g., AS, VS, and depolarization signals associated with fibrillation which are sometimes referred to as “F-waves” or “Fib-waves”) are then classified by the microcontroller 460 by comparing them to a predefined rate zone limit (i.e., bradycardia, normal, atrial tachycardia, atrial fibrillation, low rate VT, high rate VT, and fibrillation rate zones) and various other characteristics (e.g., sudden onset, stability, physiologic sensors, and morphology, etc.) in order to determine the type of remedial therapy that is needed (e.g., bradycardia pacing, antitachycardia pacing, cardioversion shocks or defibrillation shocks).

Cardiac signals are also applied to the inputs of an analog-to-digital (A/D) data acquisition system 490. The data acquisition system 490 is configured to acquire intracardiac electrogram signals, convert the raw analog data into a digital signal, and store the digital signals for later processing and/or telemetric transmission to an external device 502. The data acquisition system 490 is coupled to the right atrial lead 420, the CS lead 424, and the right ventricular lead 430 through the switch 474 to sample cardiac signals across any pair of desired electrodes. The microcontroller 460 is further coupled to a memory 494 by a suitable data/address bus 496, wherein the programmable operating parameters used by the microcontroller 460 are stored and modified, as required, in order to customize the operation of pacer/ICD 410 to suit the needs of a particular patient. Such operating parameters define, for example, pacing pulse amplitude or magnitude, pulse duration, electrode polarity, rate, sensitivity, automatic features, arrhythmia detection criteria, and the amplitude, waveshape and vector of each shocking pulse to be delivered to the patient's heart within each respective tier of therapy. Other pacing parameters include base rate, rest rate and circadian base rate.

Advantageously, the operating parameters of the implantable pacer/ICD 410 may be non-invasively programmed into the memory 494 through a telemetry circuit 500 in telemetric communication with the external device 502, such as a programmer, transtelephonic transceiver or a diagnostic system analyzer. The telemetry circuit 500 is activated by the microcontroller by a control signal 506. The telemetry circuit 500 advantageously allows intracardiac electrograms and status information relating to the operation of pacer/ICD 410 (as contained in the microcontroller 460 or memory 494) to be sent to the external device 502 through an established communication link 504. Pacer/ICD 410 further includes an accelerometer or other physiologic sensor 508, commonly referred to as a “rate-responsive” sensor because it is typically used to adjust pacing stimulation rate according to the exercise state of the patient. However, the physiological sensor 508 may further be used to detect changes in cardiac output, changes in the physiological condition of the heart, or diurnal changes in activity (e.g., detecting sleep and wake states) and to detect arousal from sleep. Accordingly, the microcontroller 460 responds by adjusting the various pacing parameters (such as rate, AV delay, V-V delay, etc.) at which the atrial and ventricular pulse generators, 470 and 472, generate stimulation pulses. While shown as being included within pacer/ICD 410, it is to be understood that the physiologic sensor 508 may also be external to pacer/ICD 410, yet still be implanted within or carried by the patient. A common type of rate responsive sensor is an activity sensor incorporating an accelerometer or a piezoelectric crystal, which is mounted within the housing 440 of pacer/ICD 410. Other types of physiologic sensors are also known, for example, sensors that sense the oxygen content of blood, respiration rate and/or minute ventilation, pH of blood, ventricular gradient, etc.

The pacer/ICD additionally includes a battery 510, which provides operating power to all of the circuits shown in FIG. 24. The battery 510 may vary depending on the capabilities of pacer/ICD 410. For pacer/ICD 410, which employs shocking therapy, the battery 510 must be capable of operating at low current drains for long periods, and then be capable of providing high-current pulses (for capacitor charging) when the patient requires a shock pulse. The battery 510 must also have a predictable discharge characteristic so that elective replacement time can be detected. Accordingly, pacer/ICD 410 is preferably capable of high voltage therapy and appropriate batteries.

As further shown in FIG. 24, pacer/ICD 410 is shown as having an impedance measuring circuit 512 which is enabled by the microcontroller 460 via a control signal 514. Thoracic impedance may be detected for use in tracking thoracic respiratory oscillations. Other uses for an impedance measuring circuit include, but are not limited to, lead impedance surveillance during the acute and chronic phases for proper lead positioning or dislodgement; detecting operable electrodes and automatically switching to an operable pair if dislodgement occurs; measuring respiration or minute ventilation; measuring thoracic impedance for determining shock thresholds; detecting when the device has been implanted; measuring respiration; and detecting the opening of heart valves, etc. The impedance measuring circuit 120 is advantageously coupled to the switch 74 so that any desired electrode may be used.

In the case where pacer/ICD 410 is intended to operate as an implantable cardioverter/defibrillator (ICD) device, it detects the occurrence of an arrhythmia, and automatically applies an appropriate electrical shock therapy to the heart aimed at terminating the detected arrhythmia. To this end, the microcontroller 460 further controls a shocking circuit 516 by way of a control signal 518. The shocking circuit 516 generates shocking pulses of low (up to 0.5 joules), moderate (0.5-10 joules) or high energy (11 to 40 joules), as controlled by the microcontroller 460. Such shocking pulses are applied to the heart of the patient through at least two shocking electrodes, and as shown in this embodiment, selected from the left atrial coil electrode 428, the RV coil electrode 436, and/or the SVC coil electrode 438. The housing 440 may act as an active electrode in combination with the RV electrode 436, or as part of a split electrical vector using the SVC coil electrode 438 or the left atrial coil electrode 428 (i.e., using the RV electrode as a common electrode). Cardioversion shocks are generally considered to be of low to moderate energy level (so as to minimize pain felt by the patient), and/or synchronized with an R-wave and/or pertaining to the treatment of tachycardia. Defibrillation shocks are generally of moderate to high energy level (i.e., corresponding to thresholds in the range of 5-40 joules), delivered asynchronously (since R-waves may be too disorganized), and pertaining exclusively to the treatment of fibrillation. Accordingly, the microcontroller 460 is capable of controlling the synchronous or asynchronous delivery of the shocking pulses.

Insofar as AF detection is concerned, the microcontroller includes an AF detector 501 for detecting AF based on ventricular channel signals using the chaos-based techniques described above. This is in addition to any atrial channel AF detection techniques. The AF detector includes, in this example, a RR interval detector 503 for detecting and tracking the aforementioned RR intervals. An irregular beat identification and elimination unit 505 eliminates ectopic beats and other irregular beats, primarily in accordance with the techniques discussed above with reference to FIGS. 19-20. A chaos-based supraventricular arrhythmia detection unit 507 detects supraventricular arrhythmias such as AF using chaos-based techniques, primarily in accordance with the procedures discussed above with reference to FIGS. 11-13 and 21. The various bins used to implement the technique may be stored in memory 494. Sudden onset evaluation unit 509 distinguishes AF from ST, primarily in accordance with the techniques discussed above with reference to FIG. 17. Microcontroller also includes components, not specifically shown, for controlling the implanted warning device 16 in response to AF. The microcontroller also controls the telemetry circuit to send appropriate warning signals via communication link 507 to the bedside monitor 18.

Not all of the microcontroller components shown need be installed within any given pacer/ICD. Also, depending upon the implementation, the various components of the microcontroller may be implemented as separate software modules or the modules may be combined to permit a single module to perform multiple functions. In addition, although shown as being components of the microcontroller, some or all of these components may be implemented separately from the microcontroller, using application specific integrated circuits (ASICs) or the like.

### Internal Components of Exemplary ICM

For the sake of completeness, internal components of ICM 20 of FIG. 3 will now be summarized with reference to FIG. 25. The housing of the device includes terminals connected to the two sensing electrodes 22, 23, which are typically mounted to the exterior surface of the housing. A programmable microcontroller 660 controls the recording of EGM signals sensed by the electrodes and diagnostics data derived therefrom. Typically, the microcontroller 660 includes the ability to process or monitor input signals (data) as controlled by a program code stored in a designated block of memory. The details of the design and operation of the microcontroller 660 are not critical to the present invention. Rather, any suitable microcontroller 660 may be used that carries out the functions described herein.

A switch 674 includes one or more switches for switchably connecting the sensing electrodes to the appropriate I/O circuits. Accordingly, the switch 674, in response to a control signal 680 from the microcontroller 660, sets the polarity of the electrodes 22, 23 by selectively closing the appropriate combination of switches (not shown). A sense amplifier 682 is coupled to the electrodes 22, 23 through switch 674 for detecting electrical cardiac activity. The switch 674 determines the “sensing polarity” of the sensed signal by selectively closing the appropriate switches, as is also known in the art. Sense amplifier 682 preferably employs a low power, precision amplifier with programmable gain and/or automatic gain control and/or automatic sensitivity control, bandpass filtering, and a threshold detection circuit, known in the art, to selectively sense electrical signals of interest.

Cardiac signals and other sensed signals are also applied to the inputs of an analog to digital (A/D) data acquisition system 690. The gain of the A/D converter 690 is controlled by the microprocessor 660 in order to match the signal amplitude and/or the resolution to a range appropriate for the function of the A/D converter 690. The data acquisition system 690 is configured to acquire subcutaneous EGM signals, convert the raw analog data into a digital signal, and store the digital signals for later processing and/or telemetric transmission to an external device 502. The microcontroller 660 is coupled to a memory 694 by a suitable data/address bus 696, wherein the programmable operating parameters used by the microcontroller 660 are stored and modified, as required, in order to customize the operation of the ICM to suit the needs of a particular patient. Such operating parameters define, for example, the threshold parameters by which AF is directed. The memory 694 includes temporary memory for temporarily storing subcutaneous EGMs in a circular buffer, as well as more permanent memory for storing EGMs correspondent to episodes of AF or other arrhythmias for later clinician review.

The operating parameters of the ICM may be non-invasively programmed into the memory 694 through a telemetry circuit 700 in telemetric communication with an external device 502, such as a programmer, transtelephonic transceiver, or a diagnostic system analyzer. The telemetry circuit 700 is activated by the microcontroller 660 by a control signal 606. The telemetry circuit 700 advantageously allows EGMs and status information relating to the operation of the ICM (as contained in the microcontroller 660 or memory 694) to be sent to the external device 502 through an established communication link 704. The telemetry circuit 700 is also responsive to commands sent via a hand-held triggering device, so as to allow the patient to trigger recording of data upon feeling any symptoms, such as heart palpitations. The ICM additionally includes a battery 710 that provides operating power to all of the circuits shown in FIG. 25.

Insofar as AF detection is concerned, the microcontroller of the ICM includes similar components to those already described with reference to FIG. 24. Namely, an AF detector 701 is provided for detecting AF based on EGM signals using the chaos-based techniques described above. The AF detector includes, in this example, a RR interval detector 703; an irregular beat identification and elimination unit 705; a chaos-based supraventricular arrhythmia detection unit 707; and a sudden onset evaluation unit 709. Microcontroller also includes components, not specifically shown, for controlling the transference of temporarily stored EGM data to more permanent memory during episodes of AF for subsequent clinician review. The microcontroller also includes components for detecting other arrhythmias, such as ventricular arrhythmias or other conditions such as syncope.

In general, while the invention has been described with reference to particular embodiments, modifications can be made thereto without departing from the spirit and scope of the invention. Note also that the term “including” as used herein is intended to be inclusive, i.e. “including but not limited to.”

What is claimed is:

1. A method for use in an implantable medical device comprising:
detecting ventricular beats and measuring intervals between the ventricular beats;
identifying irregular ventricular beats;
evaluating a degree of variability within the ventricular intervals while excluding intervals associated with the irregular beats; and
detecting a supraventricular arrhythmia based on the degree of variability.

2. The method of claim 1 wherein detecting ventricular beats is performed to detect R-waves.

3. The method of claim 2 wherein measuring intervals between the ventricular beats includes measuring RR intervals.

4. The method of claim 1 wherein identifying irregular ventricular beats includes identifying one or more of ectopic beats, bigeminal beats and trigeminal beats, premature atrial contractions (PACs), premature ventricular contractions (PVCs), and junctional beats.

5. The method of claim 1 wherein evaluating a degree of variability includes evaluating a degree of chaos within the ventricular intervals while excluding intervals associated with the irregular beats.

6. The method of claim 5 wherein evaluating the degree of chaos includes:
determining dRR values based on RR intervals of the ventricular beats while excluding RR intervals associated with the irregular beats;
determining an average dRR value;
identifying dRR values deviating significantly from the average; and
generating a numerical value representative of the number of dRR values that deviate significantly from the dRR average, the numerical value being representative of the degree of chaos.

7. The method of claim 6 wherein detecting a supraventricular arrhythmia includes:
comparing the numerical value representative of the degree of chaos against a threshold indicative of supraventricular arrhythmia; and
generating a signal indicative of an episode of supraventricular arrhythmia if the numerical value representative of the degree of chaos exceeds the threshold.

8. The method of claim 7 further including distinguishing atrial fibrillation (AF) from other supraventricular arrhythmias by:
determining whether the episode of supraventricular arrhythmia occurred substantially suddenly; and
identifying the episode as begin AF if the episode of occurred substantially suddenly and identifying the episode as being a non-AF supraventricular arrhythmias otherwise.

9. The method of claim 8 wherein non-AF supraventricular arrhythmias include sinus tachycardia (ST).

10. The method of claim 1 further including distinguishing various cardiac rhythms based on the ventricular intervals.

11. The method of claim 10 wherein distinguishing cardiac rhythms based on the ventricular intervals includes:
identifying atrial flutter and junctional rhythms based on substantially tightly varying ventricular intervals;
identifying premature atrial contractions (PACs) and premature ventricular contractions (PVCs) based on sudden changes in the ventricular intervals;
identifying bigeminal and trigeminal rhythms based on substantially cyclic variation in the ventricular intervals;
identifying sinus tachycardia (ST) based on a high degree variability in the ventricular intervals without sudden onset; and
identifying atrial fibrillation (AF) based on a high degree variability in the ventricular intervals with sudden onset.

12. An implantable medical device equipped to perform the method of claim 1.

13. The implantable medical device of claim 12 wherein the device is an implantable cardiac stimulation device.

14. The implantable medical device of claim 13 wherein the implantable cardiac stimulation device is a pacemaker.

15. The implantable medical device of claim 13 wherein the implantable cardiac stimulation device is an implantable cardioverter-defibrillator (ICD).

16. The implantable medical device of claim 12 wherein the device is an implantable cardiac monitor.

17. A system for use in an implantable medical device comprising:
a ventricular interval detector;
an irregular ventricular beat identification system;
a variability evaluation unit operative to evaluate a degree of variability within the ventricular intervals while excluding intervals associated with irregular beats; and
a supraventricular arrhythmia detector operative to detect a supraventricular arrhythmia based on the degree of variability.

18. A system for use in an implantable medical device comprising:
means for detecting ventricular beats and measuring intervals between the ventricular beats;
means for identifying irregular ventricular beats;
means for evaluating a degree of variability within the ventricular intervals while excluding intervals associated with the irregular beats; and
means for detecting a supraventricular arrhythmia based on the degree of variability.
